SEQUENCE LISTING (8 sequences)

SEQ ID NO: 1 - DNA, 22 bp, Artificial Sequence
  Synthetic Polynucleotide
catgagcacagaaagcatgatc

SEQ ID NO: 2 - DNA, 20 bp, Artificial Sequence
  Synthetic Polynucleotide
ccttctccagctggaagact

SEQ ID NO: 3 - DNA, 20 bp, Artificial Sequence
  Synthetic Polynucleotide
atggatgctaccaactggat

SEQ ID NO: 4 - DNA, 21 bp, Artificial Sequence
  Synthetic Polynucleotide
tgaaggactctggctttgtct

SEQ ID NO: 5 - DNA, 25 bp, Artificial Sequence
  Synthetic Polynucleotide
caaccaacaagtgatattctccatg

SEQ ID NO: 6 - DNA, 21 bp, Artificial Sequence
  Synthetic Polynucleotide
gatccacactctccagctgca

SEQ ID NO: 7 - DNA, 23 bp, Artificial Sequence
  Synthetic Polynucleotide
tgagcaggatggagaattacagg

SEQ ID NO: 8 - DNA, 23 bp, Artificial Sequence
  Synthetic Polynucleotide
gtccaagttcatcttctaggcac

(19) United States
(12) Patent Application Publication
(10) Pub. No.: US 2021/0161825 A1
(43) Pub. Date: Jun. 3, 2021

(54) CHLOROQUINE-BASED MATERIALS FOR THE TREATMENT OF DISEASES

(71) Applicant: BOARD OF REGENTS OF THE UNIVERSITY OF NEBRASKA, Lincoln, NE (US)

(72) Inventors: David Oupicky, La Vista, NE (US); Fei Yu, Omaha, NE (US)

(21) Appl. No.: 17/047,132

(22) PCT Filed: Apr. 12, 2019
(86) PCT No.: PCT/US19/27269

§ 371 Date: Oct. 13, 2020

Related U.S. Application Data
(60) Provisional application No. 62/657,034, filed on Apr. 13, 2018.

(51) Int. Cl. A61K 9/50; A61K 9/00; A61K 9/51; A61K 39/395; A61K 31/713; A61P 1/00; A61K 47/58; A61K 31/4706; C08F 220/28; A61K 47/69; A61K 45/06; C08F 293/00

(52) U.S. Cl. CPC A61K 9/5031; A61K 9/0053; A61K 9/5153; A61K 39/3955; A61K 31/713; A61P 1/00; C08F 2438/03; A61K 31/4706; C08F 220/281; A61K 47/6927; A61K 47/6929; A61K 45/06; C08F 293/005; A61K 47/58

(57) ABSTRACT

Provided herein are copolymers comprising a plurality of a first monomer and a plurality of a second monomer, wherein chloroquine, hydroxychloroquine, or a chloroquine analog is appended to at least a portion of the plurality of the first monomer. Also provided are nanoparticles comprising copolymers as described herein, and methods of using the copolymers and nanoparticles for treating diseases or disorders, e.g., Inflammatory Bowel Disease (IBD) or cancer (e.g., colon cancer).

### STATEMENT OF U.S. GOVERNMENT SUPPORT

This invention was made with government support under Grants No. R21EB019175 and R21EB020308 awarded by the National Institutes of Health. The government has certain rights in the invention.

### BACKGROUND

Inflammatory bowel disease (IBD) is a chronic remittent inflammatory condition of the gastrointestinal (GI) tract that manifests itself in two major forms—ulcerative colitis (UC) and Crohn's disease (CD). (Long, et al., Clin. Epidem., 5 (2013) 237-247.) UC affects the entire colon while CD involves patchy inflammation mainly in terminal ileum and colon. Despite intensive research, IBD has a poorly understood pathogenesis and no effective cure. Conventional palliative therapies which include anti-inflammatory and immunosuppressive agents represent major treatment options that can temporarily induce and maintain remission but carry significant side-effects. (Rutgeerts et al., Gut, 62 (2013) 1511-1515) Moreover, despite these available treatment options, 70% of IBD patients require at least one surgical intervention in their lifetime. (Moayyedi et al., Am. J. Gastroenterol., 106 (2011) S2-S25.)

Activation of the immune cells and production of pro-inflammatory cytokines like TNFα, IL-6, IFNγ, IL-1i are some of the hallmarks of IBD. (Cho et al., New Engl. J. Med., 361 (2009) 2066-2078.) Inhibition of the pro-inflammatory cytokines represents a promising avenue in IBD therapy. As a result, the current mainstay of IBD therapy relies on orally administered small molecule anti-inflammatory drugs and systemically given biologics. (Rogler et al., Exp. Rev. Gastroenterol. Hepatol., 9(2015) 177-189)

Chloroquine (CQ) is commonly used to prevent and treat malaria. In recent years, CQ has also found growing anti-inflammatory application in the treatment of autoimmune disorders like rheumatoid arthritis and lupus erythematosus. (Steinberg et al., Ann. Rheumatic Dis., 19 (1960) 243-250.) Several reports indicate the promise of CQ in treating IBD. (Vikramadithyan et al., Int. Immunopharmacol., 21 (2014) 328-335) However, the long-term use of CQ has severe ocular side effects like blurred vision and retinopathy. (Downes et al., Eye, 31 (2017) 972-976; Downes et al., Eye, 31 (2017) 828-845) Restricting the effect of CQ to the colon by minimizing systemic absorption is a promising strategy to enhance its local anti-inflammatory effect in IBD.

Considering the side effects that arise due to the systemic IBD treatments, oral administration is a preferred route as it can give access to inflammation specifically localized in the colon. (Friend, Adv. Drug Deliv. Rev., 57 (2005) 247-265; Keely et al., Nanomedicine: Nanotechnology, Biology and Medicine, 11 (2015) 1117-1132) Accordingly, there is a need for an orally-administered form of anti-inflammatory therapeutics such as CQ which target delivery in the lower digestive tract.

### SUMMARY

Provided herein are copolymers comprising a plurality of a first monomer and a plurality of a second monomer, wherein chloroquine, hydroxychloroquine, or a chloroquine analog is appended to at least a portion of the plurality of the first monomer. More particularly, provided are beads, microparticles, and nanoparticles comprising the copolymers, and the uses of such beads, microparticles, and nanoparticles in treating diseases or disorders, e.g., to treat IBD or cancer.

In some aspects, the disclosure provides copolymers comprising N-(2-hydroxypropyl)methacrylamide (HPMA). In other aspects, the disclosure provides copolymers further comprising a polymer block, e.g., styrene. In some aspects, the disclosure provides copolymers comprising a crosslinker, e.g., propane-2,2-diylbis(sulfanediyl))bis(propane-3,1-diyl) bis(2-methylacrylate).

Further provided herein are methods of using the copolymers disclosed to treat or prevent disease such as IBD or cancer in a subject, e.g., treating colitis or colon cancer in a subject. In some aspects, the use comprises administering to a subject a bead, microparticle, or nanoparticle comprising a copolymer disclosed herein. In some aspects, the use comprises administering the copolymer orally.

Also provided herein are methods of making the copolymers disclosed, comprising admixing a plurality of the first monomer and the second monomer under conditions to polymerize the first monomer and the second monomer to form the copolymer, optionally in the presence of a polymerization initiator, a chain transfer agent, or both. In some aspects, the method comprises admixing in the presence of a polymerization initiator, e.g., AIBN. In other aspects, the method comprises admixing in the presence of a chain transfer agent, e.g., (4-cyano-4-(phenyl-carbonothioylthio)pentanoic acid), (4-cyano-4-[(ethylsulfanylthiocarbonyl)sulfanyl]pentanoic acid), (4-cyano-4-[(dodecyl sulfanylthiocarbonyl)sulfanyl]pentanoic acid), (2-cyano-2-propyl dodecyl trithiocarbonate), or a mixture thereof.

Other aspects of the disclosure include a copolymer or nanoparticle as disclosed herein for use in the preparation of a medicament for treating or preventing a disease or disorder in a subject, and the use of a compound as disclosed herein in a method of treating or preventing a disease or disorder in a subject.

### BRIEF DESCRIPTION OF THE FIGURES

FIG. 1 shows gel permeation chromatographs of (A) alkyne-containing poly(2-hydroxypropyl methacrylamide) polymers (pHP) prepared using 2-cyano-2 propyl dodecyl trithiocarbonate (left to right: pHP20H, pHP20M, pHP10, pHP5, pHP0, pHP20, pHP20L, and pHP40), (B) poly(2-hydroxypropyl methacrylamide) polymers “clicked” to CQ (pHP-CQ) (left to right: pHP20H-CQ, pHP20M-CQ, pHP10-CQ, pHP20-CQ, pHP20L-CQ, pHP5-CQ, pHP40-CQ, and pHP0), and (C) alkyne-containing poly(2-hydroxypropyl methacrylate) polymers (pHPte) prepared using 4-cyano-4-[(ethylsulfanylthiocarbonyl)sulfanyl]pentanoic acid (left to right: pHPte20H, pHPte20M, pHPte20, pHPte10, pHPte40, pHPte5, pHPte0, and pHPte20L).

FIG. 2 shows a comparison between the gel permeation chromatographs of poly(HPMA-co-PPMA) before (pHP) and after (pHP-CQ) “clicking” of CQ-N₃ to the polymer-(A) pHP5-CQ (left) and pHP5 (right), (B) pHP10-CQ (left) and pHP10 (right), (C) pHP20-CQ (left) and pHP20 (right), (D) pHP40-CQ (left) and pHP40 (right), (E) pHP20L-CQ (left) and pHP20L (right), (F) pHP20M-CQ (left) and pHP20M (right), and (G) pHP20H-CQ (left) and pHP20H (right).

FIG. 3 shows an analysis of C. rodentium colitis. Mice were gavaged with 5×10⁸ CFU of C. rodentium and sacrificed on day 1, 10, and 14. (a) Cytokine levels in the colon homogenates were analyzed using Luminex ProcartaPlex panel. Data are represented as mean fold change vs. healthy animals. (b) Representative images of colon. (c) Colon weight.

FIG. 4 shows the blood concentration vs. time profile in mice subjected to the C. rodentium colitis orally administered HCQ or pCQ (30 mg/kg CQ equivalent). Results are shown as HCQ blood concentration±SD (n=3). Data shown represent the total (polymer-bound plus free) HCQ blood concentration.

FIG. 5 shows liver and colon distribution of pCQ in colitis mice. Animals treated with a single oral dose of pCQ and HCQ (30 mg/kg CQ equivalent) were sacrificed at pre-determined time points and total HCQ levels measured in liver and colon. Results are shown as concentration of HCQ in the tissues±SD (n=3). pCQ data show the total (polymer-bound+free) HCQ blood concentration.

FIG. 6 shows hydrolysis of pCQ in the colon. Concentration of polymer-bound and free HCQ were measured in colons from colitis mice. Results are expressed as HCQ conc.±SD (n=3).

FIG. 7 shows liver (a) and colon (b) metabolism of pCQ and HCQ. Tissue homogenates were analyzed for metabolites. Data are represented as mean metabolite concentration in ng/g of tissue±SD.

FIG. 8 shows the effect of oral pCQ on colon inflammation. Mice with C. rodentium colitis were orally administered with seven pCQ or HCQ doses (30 mg/kg CQ). (a) Representative images of H&E stained colon; (b) colon crypt length; (c) histopathological score. Data are represented as mean±SD (n=5).

FIG. 9 shows the effect of pCQ on macrophage infiltration in the colon. (a) Representative images of CD68-stained colon tissue slides; (b) Quantitation of the CD68-positive cells. Data are represented as mean CD68 positive cells per HPF±SD.

FIG. 10 shows the effect of pCQ on epithelial apoptosis. (a) Representative images of CC3 stained colon tissue slides; (b) quantitation of CC3-positive cells. Data are represented as mean CC3 positive cells/colon section±SD.

FIG. 11 shows the effect of pCQ on colon cytokine mRNA levels. Fold change in colon mRNA expression relative to healthy control (a) TNF-a; (b) IL-6; (c) IL-1P; and (d) IL-2. Data are represented as mean±SD (n=5).

FIG. 12 shows the effect of pCQ on STAT-3 expression. Representative images of STAT-3 stained colon tissue slides (larger panel: 20× magnification, smaller panel: 40× magnification).

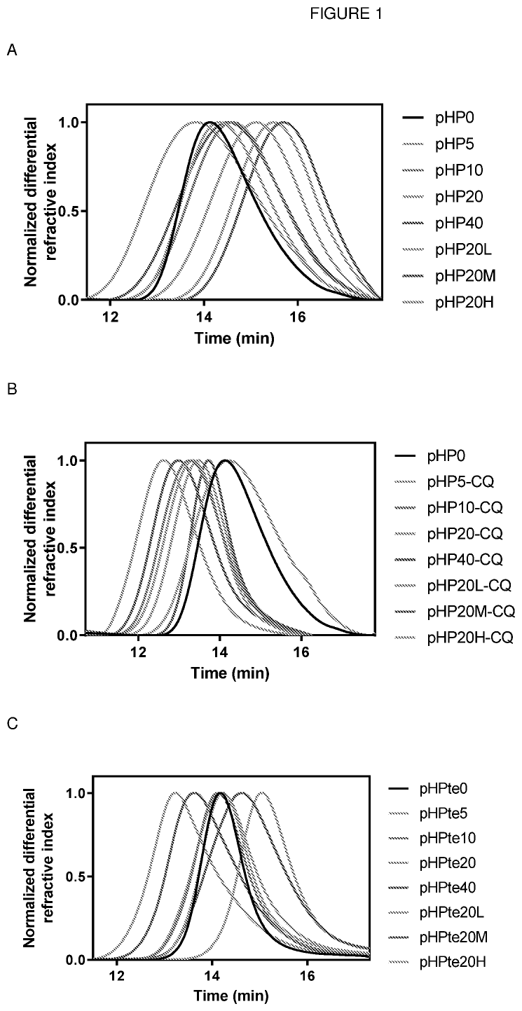

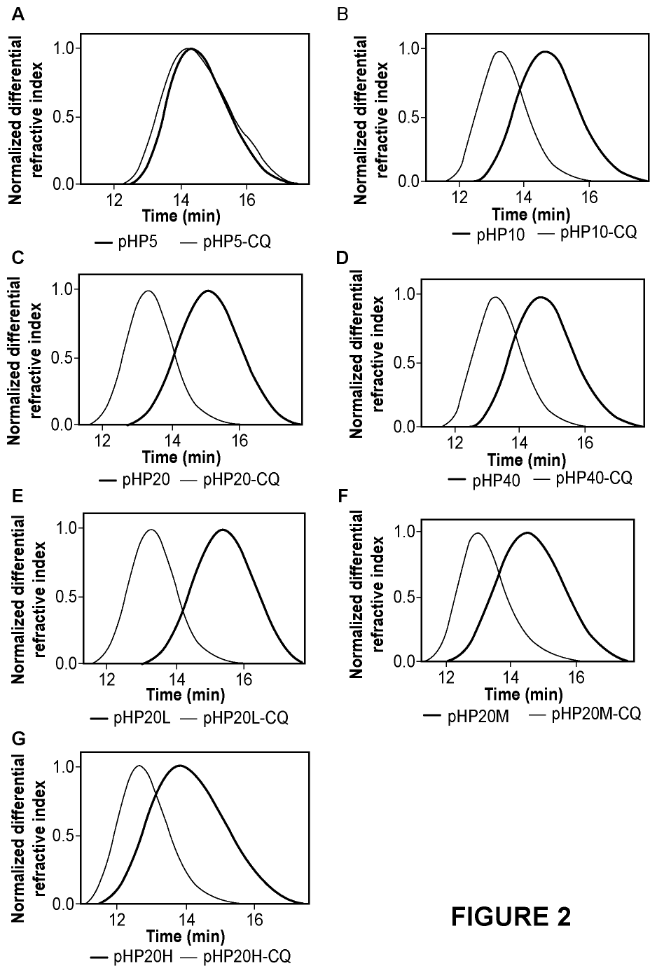

### DETAILED DESCRIPTION

The present disclosure provides compositions and methods for the treatment of diseases and disorders, such as inflammatory bowel disease (IBD) and related disorders. In particular, provided are chloroquine and hydroxychloroquine-based polymers which may be formed into beads, microparticles, and/or nanoparticles. The beads, microparticles and/or nanoparticles may also be used to package other drugs for the treatment of diseases or disorders, e.g., IBD.

The main challenge in the development of oral IBD therapies is the physiological changes in the course of the disease. (Keely et al., Nanomedicine: Nanotechnology, Biology and Medicine, 11 (2015) 1117-1132.) The GI environment is diverse and factors like residence time, changes in pH, enzymatic or microbial degradation play an important role in drug delivery. These factors vary significantly between IBD patients and healthy individuals. For example, colonic transit time ranges from 6 to 70 hours in IBD patients as compared to 4 to 6 hours in healthy individuals. (Yosida et al., Pharm. Res., 17 (2000) 160-167; Wilding et al., Pharm. Res., 8 (1991) 360-364.) Colon pH in IBD patients is significantly lower than in healthy colon. (Rasmussen et al., Digest. Dis. Sci., 38 (1993) 1989-1993; Evans et al., Gut, 48 (2001) 571.) The epithelial barrier represents one of the tightest barriers in the human body. (Nusrat et al., World J. Gastroenterol., 14 (2008) 401-407.) It consists of tight junction proteins, which selectively regulate the transport across the colonic epithelium. Inflammation alters the permeability of this barrier and consequently, the protective mechanisms significantly alter the physiology of the colonic epithelium, which in turn affects drug transport.

Due to the chronic nature of IBD, patients remain on various treatments for life and thus long-term effects of the therapy represent a significant concern. Various systemic therapies have been explored, but the systemic effects of long-term anti-inflammatory treatment remains a concern.

Many strategies have been explored for local oral drug delivery to the inflamed colon. Conventional delivery systems are designed to function well in healthy intestinal pH but may perform poorly in IBD. (Keely et al., Nanomedicine: Nanotechnology, Biology and Medicine, 11 (2015) 1117-1132; Aggarwal et al., Drug Deliv. Transl. Res., 4 (2014) 187-202.) Nanoparticles and microparticles have been researched with respect to their size as well as charge for accumulation in inflamed colon. Results have shown that passively targeted systems with particle size ranging from 100 to 300 nm and positive surface charge preferentially accumulate in the inflamed colon. (Lehr et al., J. Controlled Rel., 161 (2012) 235-246; Rubinstein et al., Mol. Pharm., 6 (2009) 1083-1091.) The specificity of these systems can be enhanced by incorporation of targeting ligands (e.g. lectins, mannose) that bind receptors overexpressed in the inflamed colon. (Merlin et al., Biomaterials, 34 (2013) 7471-7482; Merlin et al., Gastroenterol., 146 (2014) 1289-1300.e1219.)

Conjugating small molecule drugs to polymers has been explored as another delivery strategy to enhance colon retention. (Vicent et al., Nanomedicine, 5 (2010) 915-935.) Dexamethasone-poly(dimethylamino)ethyl methacrylate conjugates and dextran-budesonide conjugates have been reported as potential orally administered alternatives to free dexamethasone and budesonide that overcome systemic side effects and pH lability, respectively. (Dorkoosh et al., Int. J. Pharm., 365 (2009) 69-76; Brayden et al., J. Controlled Rel., 135 (2009) 35-43.)

Thus, provided herein are copolymers comprising chloroquine, hydroxychloroquine, or a chloroquine analog that can treat or prevent a disease or disorder associated with inflammation in a subject. These copolymers are useful in the treatment of a variety of diseases and disorders, including but not limited to, IBD and cancer. Particularly, they allow treatment to be targeted directly to the lower gastrointestinal tract, without the need for systemic therapy.

### Copolymers of the Disclosure

Provided herein are copolymers comprising a plurality of a first monomer and a plurality of a second monomer, wherein chloroquine, hydroxychloroquine, or a chloroquine analog is appended to at least a portion of the plurality of the first monomer. In some cases, chloroquine, hydroxychloroquine, or a chloroquine analog is appended to each of the first monomer. In some cases, chloroquine, hydroxychloroquine, or a chloroquine analog is appended to a portion of the first monomer. In some cases, the copolymer comprises chloroquine. In some cases, the copolymer comprises hydroxychloroquine. In some cases, the copolymer comprises a chloroquine analog.

In some cases, the chloroquine analog is hydroxychloroquine, methacryloyl chloroquine, quinacrine, 8-hydroxyquinoline, primaquine, sontoquine, azidoquine, or methacryloyl triazole chloroquine. In some cases, the chloroquine analog is methacryloyl chloroquine.

In some cases, at least one of the first monomer and the second comprises an acrylate, an acrylamide, or an alkene. In some cases, the first monomer having chloroquine appended thereto comprises

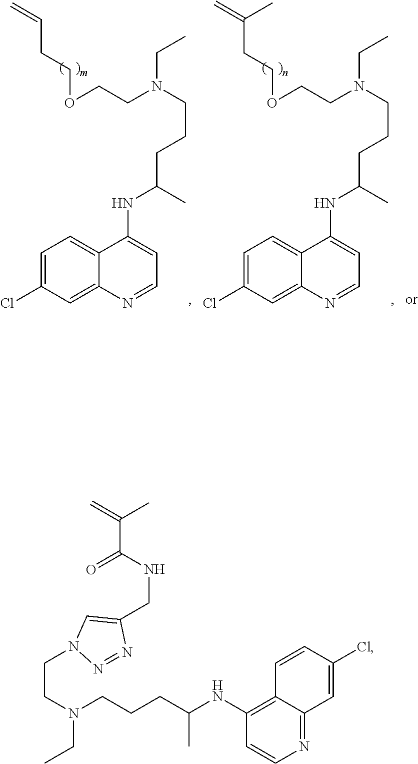

wherein m and n each are independently an integer from 0 to 10.

In some cases, the first monomer having chloroquine appended thereto comprises

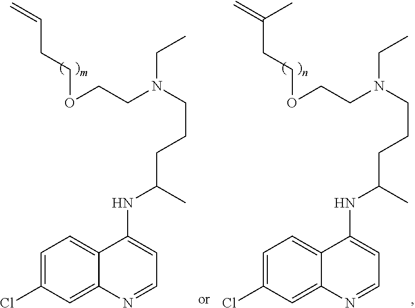

where m and n are each independently an integer from 0 to 10 (e.g., 0, 1, 2, 3, 4, 5, 6, 7, 8, 9, or 10), and in some cases, m or n is 0, 1, or 2. In some cases, the first monomer having chloroquine appended thereto comprises

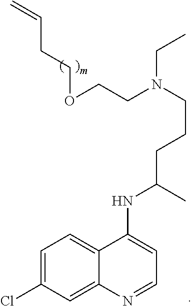

In some cases, m is 0. In some cases, m is 1. In some cases, m is 2. In some cases, m is 3. In some cases, m is 4. In some cases, m is 5. In some cases, m is 6. In some cases, m is 7. In some cases, m is 8. In some cases, m is 9. In some cases, m is 10. In some cases, the first monomer having chloroquine appended thereto comprises

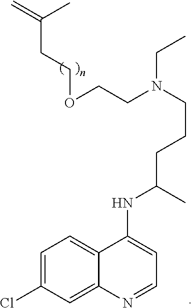

In some cases, n is 0. In some cases, n is 1. In some cases, n is 2. In some cases, n is 3. In some cases, n is 4. In some cases, n is 5. In some cases, n is 6. In some cases, n is 7. In some cases, n is 8. In some cases, n is 9. In some cases, n is 10. In some cases, the first monomer having chloroquine appended thereto comprises

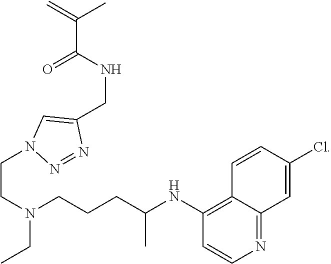 [SMILES: C=C(C)C(=O)NCc1cn(CCN(CC)CCCC(C)Nc2ccnc3cc(Cl)ccc23)nn1]

In some cases, the second monomer comprises an acrylamide monomer. In some cases, the second monomer comprises N-(2-hydroxypropyl)methacrylamide (HPMA).

In some cases, the copolymer further comprises a polymer block. In some cases, the polymer block comprises poly(lactide-co-glycolide) (PLGA), polylactic acid (PLA), polycaprolactone (PCL), other polyesters, poly(propylene oxide), poly(1,2-butylene oxide), poly(n-butylene oxide), poly(tetrahydrofurane), and polystyrene; glyceryl esters, polyoxyethylene fatty alcohol ethers, polyoxyethylene sorbitan fatty acid esters, polyoxyethylene fatty acid esters, sorbitan esters, glycerol monostearate, polyethylene glycols, polypropyleneglycols, cetyl alcohol, cetostearyl alcohol, stearyl alcohol, aryl alkyl polyether alcohols, polyoxyethylene-polyoxypropylene copolymers, poloxamines, cellulose, methylcellulose, hydroxylmethylcellulose, hydroxypropylcellulose, hydroxypropylmethylcellulose, polysaccharides, starch and their derivatives, hydroxyethylstarch, polyvinyl alcohol (PVA), polyvinylpyrrolidone phospholipids, amphiphilic lipids, 1,2-dialkylglycero-3-alkylphophocholines, 1,2-distearoyl-sn-glecro-3-phosphocholine (DSPC), 1,2-distearoyl-sn-glycero-3-phosphoethanolamine-N-[carboxy(polyethylene glycol) (DSPE-PEG), dimethylaminoethanecarbamoyl cheolesterol (DC-Chol), N-[1-(2,3-Dioleoyloxy)propyl]-N,N,N-trimethylammonium (DOTAP), alkyl pyridinium halides, quaternary ammonium compounds, lauryldimethylbenzylammonium, acyl carnitine hydrochlorides, dimethyldioctadecylammonium (DDAB), n-octylamines, oleylamines, benzalkonium, cetyltrimethylammonium, chitosan, chitosan salts, poly(ethylenimine) (PEI), poly(N-isopropyl acrylamide (PNIPAM), and poly(allylamine) (PAH), poly (dimethyldiallylammonium chloride) (PDDA), alkyl sulfonates, alkyl phosphates, alkyl phosphonates, potassium laurate, triethanolamine stearate, sodium lauryl sulfate, sodium dodecylsulfate, alkyl polyoxyethylene sulfates, alginic acid, alginic acid salts, hyaluronic acid, hyaluronic acid salts, gelatins, dioctyl sodium sulfosuccinate, sodium carboxymethylcellulose, cellulose sulfate, dextran sulfate and carboxymethylcellulose, chondroitin sulfate, heparin, synthetic poly(acrylic acid) (PAA), poly (methacrylic acid) (PMA), poly(vinyl sulfate) (PVS), poly(styrene sulfonate) (PSS), esters and amides of poly(methacrylic acid) and poly(acrylic acid) (e.g., alkylmethacrylates—methyl, ethyl, propyl, butyl) bile acids and their salts, cholic acid, deoxycholic acid, glycocholic acid, taurocholic acid, glycodeoxycholic acid, a derivative thereof, or a combination thereof. In some cases, the polymer block comprises styrene.

In some cases, the second monomer comprises HPMA and the polymer block comprises polystyrene.

In some cases, the chloroquine, hydroxychloroquine, or chloroquine analog is attached to the first monomer via a bond cleavable from the co-polymer under physiological conditions. Some examples of bonds cleavable under physiological conditions include disulfide, ester, ether, amide, imine, acetal, thioketal, orthothioester, and orthoester bonds.

In some cases, the chloroquine, hydroxychloroquine, or chloroquine analog is attached to the first monomer via an amide bond, an ester bond, a 5-10 membered heterocycle, or a peptide bond. In some cases, the chloroquine, hydroxychloroquine, or chloroquine analog is attached to the first monomer via an amide bond. In some cases, the chloroquine, hydroxychloroquine, or chloroquine analog is attached to the first monomer via an ester bond. In some cases, the chloroquine, hydroxychloroquine, or chloroquine analog is attached to the first monomer via a 5-10 membered heterocycle. In some cases, the chloroquine, hydroxychloroquine, or chloroquine analog is attached to the first monomer via a 5-membered heterocycle. In some cases, the chloroquine, hydroxychloroquine, or chloroquine analog is attached to the first monomer via a triazolyl ring (e.g., the result of a “click” chemistry coupling of an alkyne and an azide).

In some cases, the chloroquine, hydroxychloroquine, or chloroquine analog is attached to the first monomer via a bond not cleavable under physiological conditions. In some cases, the choroquine, hydroxychloroquine, or chloroquine analog is attached to the first monomer via a triazolyl ring.

Some specifically contemplated copolymers described herein have the structure of:

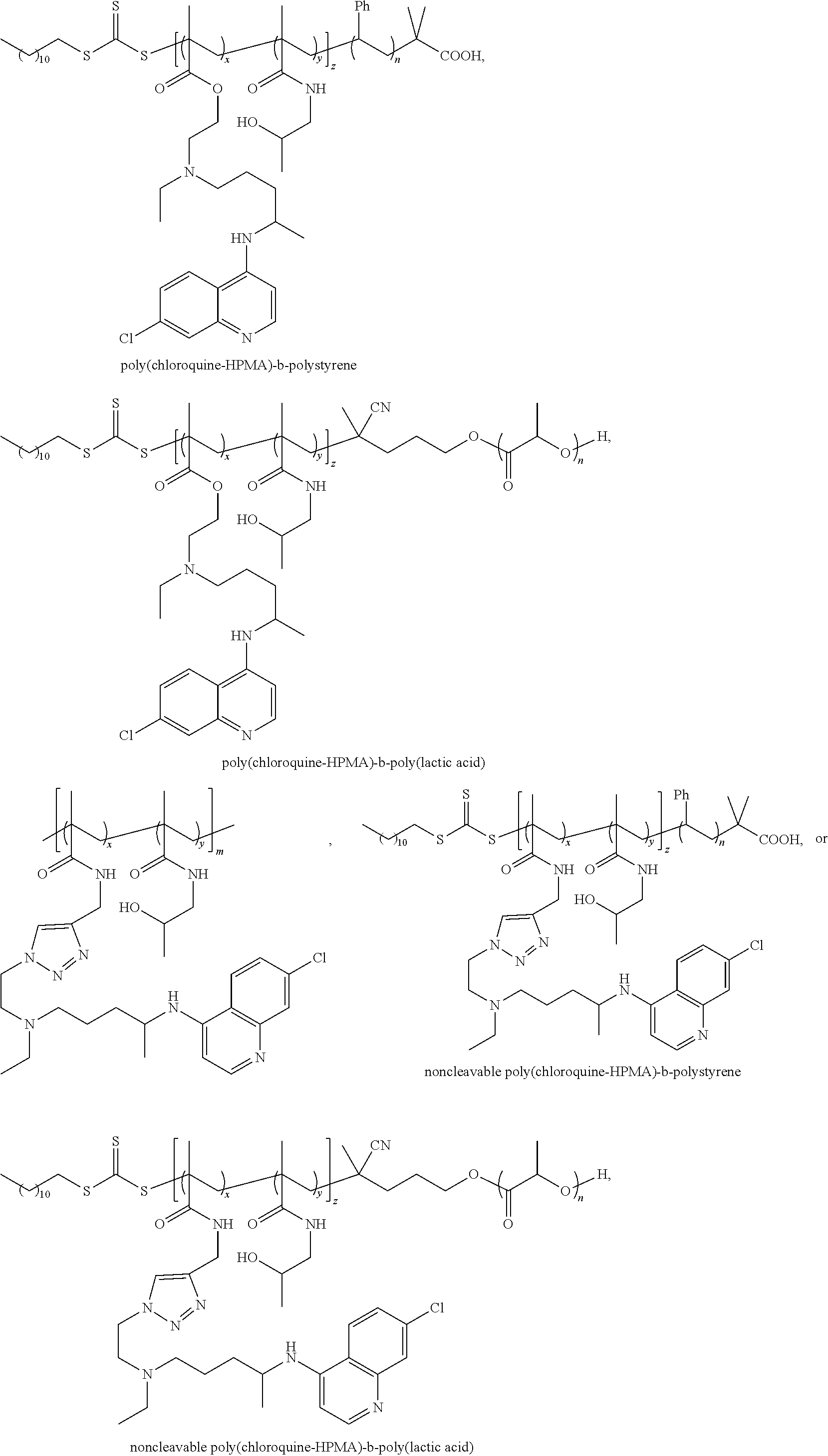

wherein each x is independently an integer of 1 to 200, each y is independently an integer of 0 to 500, each z is independently an integer of 1 to 500, each m is independently an integer of 1 to 500, and each n is independently an integer of 1 to 10,000.

In some cases, the copolymer has the structure of

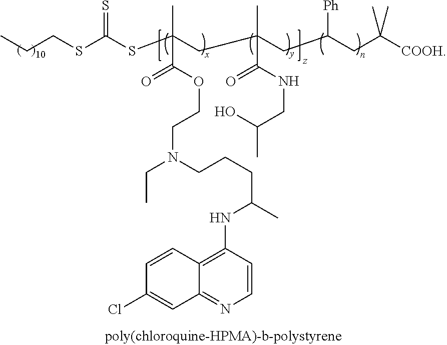

In some cases, the copolymer has the structure of

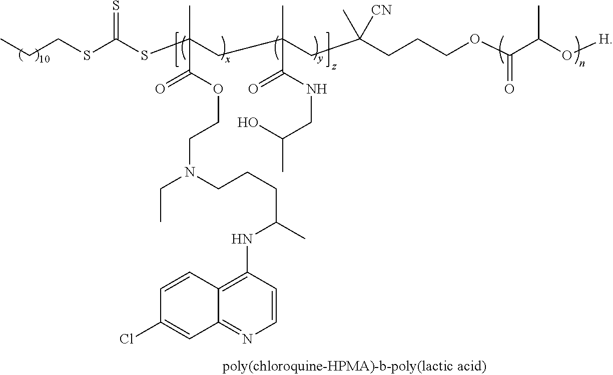

In some cases, the copolymer has the structure of

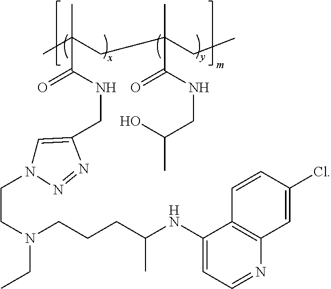

In some cases, the copolymer has the structure of

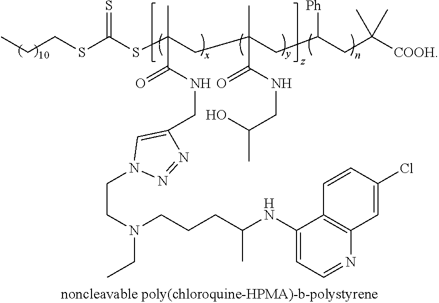

In some cases, the copolymer has the structure of

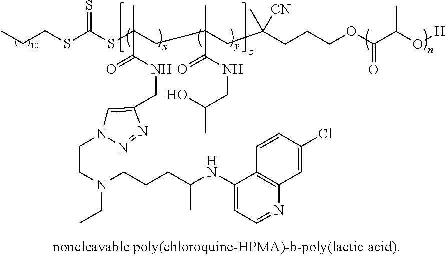

In some cases, the copolymers disclosed herein comprise a crosslinker. In some cases, the crosslinker is cleavable under physiological conditions. In some cases, the crosslinker is a reactive oxygen species—(ROS)—sensitive linker. In some cases, the ROS-sensitive linker comprises a thioketal crosslinker, organochalcogen, arylboronic esters, thioether, vinyldithioether, aryl oxalate ester, or ferrocene. In some cases, the crosslinker is enzymatically-degradable. In some cases, the crosslinker is an aromatic azo linker degradable by colonic azo-reductases. In some cases, the crosslinker comprises

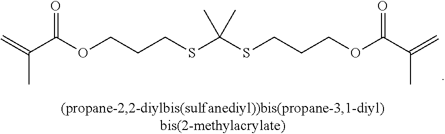 [SMILES: C=C(C)C(=O)OCCCSC(C)(C)SCCCOC(=O)C(=C)C]

In some cases, the disclosed co-polymers are further conjugated to a polymer block. In some cases, the copolymers are attached to copolymers of N-(2-hydroxypropyl)methacrylamide (HPMA). In various embodiments, the copolymers are attached to copolymers of hydroxypropyl methacrylate. In some embodiments, the co-polymers are attached to copolymers of 2-(dimethlamino)ethyl methacrylate.

Non-limiting examples of chloroquine, hydroxychloroquine, or chloroquine analogs include chloroquine, hydroxychloroquine, methacryloyl chloroquine, quinacrine, 8-hydroxyquinoline, primaquine, sontoquine, and azidochloroquine, and methacryloyl triazole chloroquine. In some cases, the chloroquine, hydroxychloroquine, or chloroquine analog is cleavable from the copolymer. In some cases, the chloroquine, hydroxychloroquine, or chloroquine analog is not cleavable from the copolymer.

In various embodiments the co-polymer (e.g., a chloroquine-HPMA co-polymer) can be combined with another polymer block as shown in Scheme 1a.

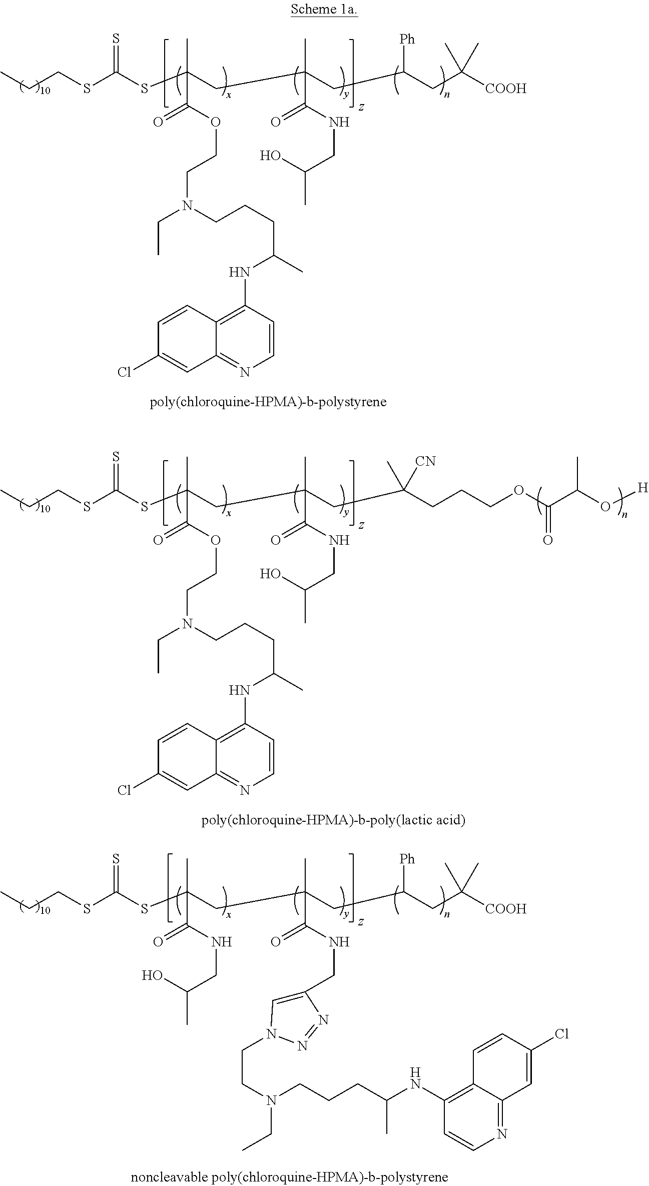

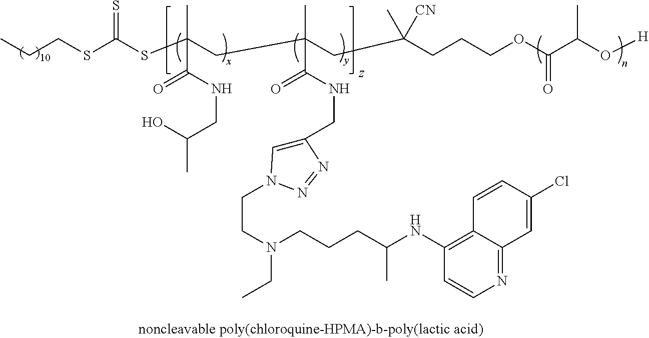

Non-limiting examples of suitable polymers which can comprise the polymer block are poly(lactide-co-glycolide) (PLGA), polylactic acid (PLA), polycaprolactone (PCL), other polyesters, poly(propylene oxide), poly(1,2-butylene oxide), poly(n-butylene oxide), poly(tetrahydrofurane), and polystyrene; glyceryl esters, polyoxyethylene fatty alcohol ethers, polyoxyethylene sorbitan fatty acid esters, polyoxyethylene fatty acid esters, sorbitan esters, glycerol monostearate, polyethylene glycols, polypropyleneglycols, cetyl alcohol, cetostearyl alcohol, stearyl alcohol, aryl alkyl polyether alcohols, polyoxyethylene-polyoxypropylene copolymers, poloxamines, cellulose, methylcellulose, hydroxylmethylcellulose, hydroxypropylcellulose, hydroxypropylmethylcellulose, polysaccharides, starch and their derivatives, hydroxyethylstarch, polyvinyl alcohol (PVA), polyvinylpyrrolidone phospholipids, amphiphilic lipids, 1,2-dialkylglycero-3-alkylphophocholines, 1,2-distearoyl-sn-glecro-3-phosphocholine (DSPC), 1,2-distearoyl-sn-glycero-3-phosphoethanolamine-N-[carboxy(polyethylene glycol) (DSPE-PEG), dimethylaminoethanecarbamoyl cheolesterol (DC-Chol), N-[1-(2,3-Dioleoyloxy)propyl]-N,N,N-trimethylammonium (DOTAP), alkyl pyridinium halides, quaternary ammonium compounds, lauryldimethylbenzylammonium, acyl carnitine hydrochlorides, dimethyldioctadecylammonium (DDAB), n-octylamines, oleylamines, benzalkonium, cetyltrimethylammonium, chitosan, chitosan salts, poly(ethylenimine) (PEI), poly(N-isopropyl acrylamide (PNIPAM), and poly(allylamine) (PAH), poly (dimethyldiallylammonium chloride) (PDDA), alkyl sulfonates, alkyl phosphates, alkyl phosphonates, potassium laurate, triethanolamine stearate, sodium lauryl sulfate, sodium dodecylsulfate, alkyl polyoxyethylene sulfates, alginic acid, alginic acid salts, hyaluronic acid, hyaluronic acid salts, gelatins, dioctyl sodium sulfosuccinate, sodium carboxymethylcellulose, cellulose sulfate, dextran sulfate and carboxymethylcellulose, chondroitin sulfate, heparin, synthetic poly(acrylic acid) (PAA), poly (methacrylic acid) (PMA), poly(vinyl sulfate) (PVS), poly(styrene sulfonate) (PSS), esters and amides of poly(methacrylic acid) and poly(acrylic acid) (e.g., alkylmethacrylates—methyl, ethyl, propyl, butyl) bile acids and their salts, cholic acid, deoxycholic acid, glycocholic acid, taurocholic acid, glycodeoxycholic acid, derivatives thereof, and combinations thereof. In one embodiment the additional polymer blocks comprise polystyrene.

### Beads, Microparticles, and Nanoparticles of the Disclosure

Also provided are beads, microparticles and nanoparticles comprising a plurality of the copolymers described herein. In some cases, the bead, microparticle or nanoparticle comprises a copolymer further having a polymer block. In some cases, the bead, microparticle or nanoparticle comprises a polymer block comprising poly(lactide-co-glycolide) (PLGA), polylactic acid (PLA), polycaprolactone (PCL), other polyesters, poly(propylene oxide), poly(1,2-butylene oxide), poly(n-butylene oxide), poly(tetrahydrofurane), and polystyrene; glyceryl esters, polyoxyethylene fatty alcohol ethers, polyoxyethylene sorbitan fatty acid esters, polyoxyethylene fatty acid esters, sorbitan esters, glycerol monostearate, polyethylene glycols, polypropyleneglycols, cetyl alcohol, cetostearyl alcohol, stearyl alcohol, aryl alkyl polyether alcohols, polyoxyethylene-polyoxypropylene copolymers, poloxamines, cellulose, methylcellulose, hydroxylmethylcellulose, hydroxypropylcellulose, hydroxypropylmethylcellulose, polysaccharides, starch and their derivatives, hydroxyethylstarch, polyvinyl alcohol (PVA), polyvinylpyrrolidone phospholipids, amphiphilic lipids, 1,2-dialkylglycero-3-alkylphophocholines, 1,2-distearoyl-sn-glecro-3-phosphocholine (DSPC), 1,2-distearoyl-sn-glycero-3-phosphoethanolamine-N-[carboxy(polyethylene glycol) (DSPE-PEG), dimethylaminoethanecarbamoyl cheolesterol (DC-Chol), N-[1-(2,3-Dioleoyloxy)propyl]-N,N,N-trimethylammonium (DOTAP), alkyl pyridinium halides, quaternary ammonium compounds, lauryldimethylbenzylammonium, acyl carnitine hydrochlorides, dimethyldioctadecylammonium (DDAB), n-octylamines, oleylamines, benzalkonium, cetyltrimethylammonium, chitosan, chitosan salts, poly(ethylenimine) (PEI), poly(N-isopropyl acrylamide (PNIPAM), and poly(allylamine) (PAH), poly (dimethyldiallylammonium chloride) (PDDA), alkyl sulfonates, alkyl phosphates, alkyl phosphonates, potassium laurate, triethanolamine stearate, sodium lauryl sulfate, sodium dodecylsulfate, alkyl polyoxyethylene sulfates, alginic acid, alginic acid salts, hyaluronic acid, hyaluronic acid salts, gelatins, dioctyl sodium sulfosuccinate, sodium carboxymethylcellulose, cellulose sulfate, dextran sulfate and carboxymethylcellulose, chondroitin sulfate, heparin, synthetic poly(acrylic acid) (PAA), poly (methacrylic acid) (PMA), poly(vinyl sulfate) (PVS), poly(styrene sulfonate) (PSS), esters and amides of poly(methacrylic acid) and poly(acrylic acid) (e.g., alkylmethacrylates—methyl, ethyl, propyl, butyl) bile acids and their salts, cholic acid, deoxycholic acid, glycocholic acid, taurocholic acid, glycodeoxycholic acid, a derivative thereof, or a combination thereof.

In some cases, the bead, microparticle or nanoparticle further comprises a therapeutic agent. In some cases, the therapeutic agent comprises an anti-inflammatory compound, an immune system suppressor, an antibiotic, or a combination thereof. In some cases, the anti-inflammatory compound comprises an aminosalicylate, a corticosteroid, cyclosporine, small interfering RNA (siRNA), or an antibody. In some cases, the antibody or siRNA is an anti-TNFα antibody or anti-TNFα siRNA.

In some cases, the bead, microparticle, or nanoparticle further comprises a targeting ligand. In some cases, the targeting ligand comprises a lectin or mannose. In some cases, the targeting ligand comprises a lectin. In some cases, the targeting ligand comprises mannose.

In some embodiments, the co-polymers disclosed herein are formed into beads, microparticles, and/or nanoparticles by incorporating additional polymer blocks into the co-polymer to aid in bead, microparticle, and/or nanoparticle formation (e.g. polystyrene blocks). In some cases, the co-polymers with or without additional polymer blocks (e.g. polystyrene blocks) are linked together using a linker group. In some embodiments, the linker group is biologically cleavable linker group. In some embodiments, the linker group is a reactive oxygen species (ROS) sensitive linker group. Nonlimiting examples of ROS sensitive linkers include but are not limited to thioketal cross-linkers, organochalcogen (C—Se, Se—Se or C—Te, Te—Te), arylboronic esters, thioether, vinyldithioether, aryl oxalate ester, and ferrocene. In some embodiments, the ROS sensitive linker group is a thioketal cross-linker. Without wishing to be bound by theory, the linker group allows for the individual polymer strands to be aggregated together to form a bead, microparticle, and/or nanoparticle. In some cases, the linker can be cleaved to release the individual particle strands upon delivery to the area of inflammation within the body (e.g. intestine). In some cases, the beads, microparticles, and/or nanoparticles are modified with various targeting moieties including but not limited to antibodies, proteins, peptides, and small molecules to target the compositions to specific areas of disease. In some cases, beads, microparticles, and nanoparticles described herein can be encapsulated or coated (e.g., by spray-drying) with various enteric coatings (e.g., Eudragit) to protect them from degradation in vivo (e.g., in the stomach).

In embodiments, beads, microparticles, and/or nanoparticles can be used to deliver additional therapeutics to help treat diseases or disorders, e.g., IBD. In some cases, beads, microparticles, and/or nanoparticles described herein can be used to encapsulate/deliver additional therapeutics including but not limited to anti-inflammatory compounds, immune system suppressors, antibiotics, and combinations thereof. In some cases, the anti-inflammatory compounds include but are not limited to corticosteroids and aminosalicylates (ex. Mesalamine, balsalazide, and olsalazine). In some cases, the immune system suppressors include but are not limited to azathioprine, mercaptopurine, cyclosporine, methotrexate, infliximab, adalimumab, golimumab, natalizumab, vedolizumab, and ustekinumab. In some cases, the antibiotics include but are not limited to ciprofloxacin and metronidazole. In some cases, the additional therapeutic or therapeutics can be co-administered with co-polymers as disclosed herein that are not formed into micro and/or nanoparticles. In some cases, the additional therapeutic or therapeutics can be administered separately from co-polymers as disclosed herein that are not formed into micro and/or nanoparticles. In some cases, the additional therapeutic or therapeutics can be co-administered with co-polymers as disclosed herein that are formed into beads, microparticles, and/or nanoparticles. In some cases, the additional therapeutic or therapeutics can be administered separately from co-polymers as disclosed herein that are formed into beads, microparticles, and/or nanoparticles.

### Synthesis of Chloroquine Copolymers of the Disclosure

The copolymers disclosed herein can be prepared in a variety of ways using commercially available starting materials, intermediates as reported in the literature, or from readily prepared intermediates, by employing standard synthetic methods and procedures either known to those skilled in the art, or in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T.W., Wuts, P.G.M., Protective Groups in Organic Synthesis, 3^{rd} edition, John Wiley & Sons: New York, 1999, are useful and recognized reference textbooks of organic synthesis known to those in the art. The following descriptions of synthetic methods are designed to illustrate, but not to limit, general procedures for the preparation of compounds and copolymers of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt, ester or prodrug thereof.

Disclosed herein are methods of making copolymers described herein. In some cases, the method comprises admixing a plurality of a first monomer and a second monomer under conditions to polymerize the first monomer and the second monomer to form the copolymer, optionally in the presence of a polymerization initiator, a chain transfer agent, or both. In some cases, the method comprises admixing in the presence of a polymerization initiator. In some cases, the method comprises admixing in the presence of a chain transfer agent. In some cases, the method comprises admixing in the presence of both a polymerization initiator and a chain transfer reagent. In some cases, the polymerization initiator comprises azobisisobutyronitrile (AIBN). In some cases, the chain transfer agent comprises

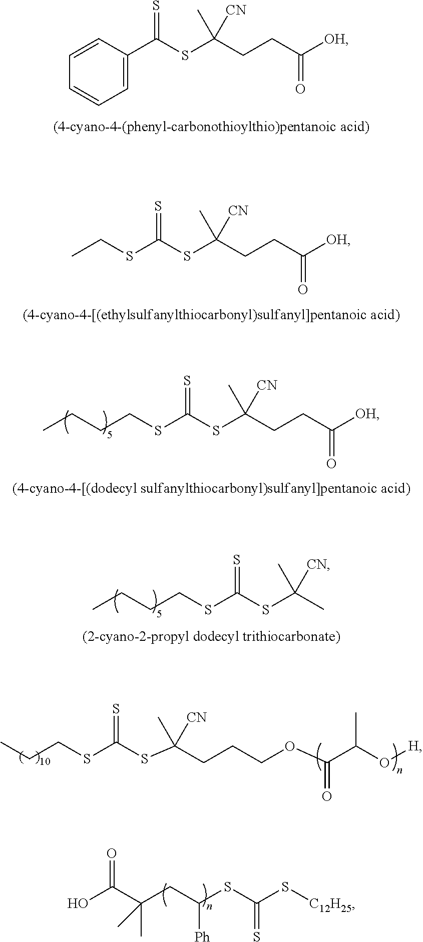

or a mixture thereof. In some cases, the chain transfer agent comprises

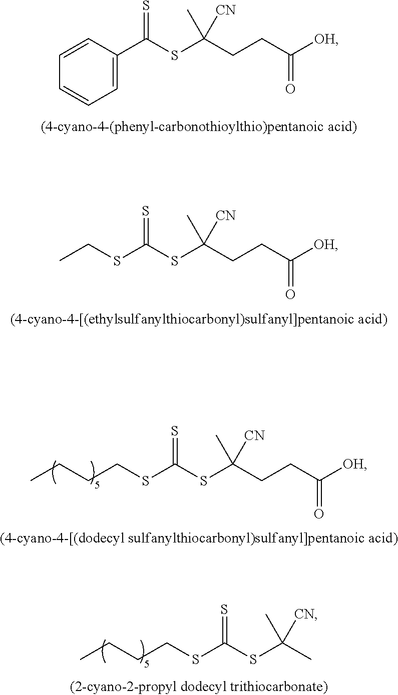

or a mixture thereof. In some cases, the chain transfer agent comprises

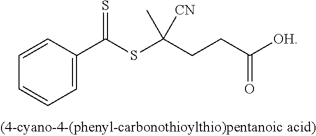 [SMILES: CC(C#N)(CCC(=O)O)SC(=S)c1ccccc1]

In some cases, the chain transfer agent comprises

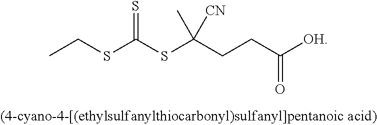

In some cases, the chain transfer agent comprises

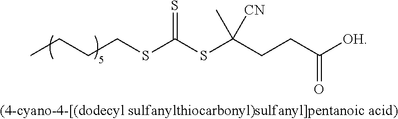

In some cases, the chain transfer agent comprises

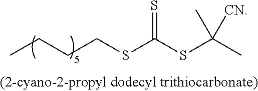

In some cases, the chain transfer agent comprises

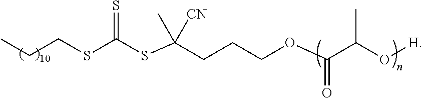

In some cases, the chain transfer agent comprises

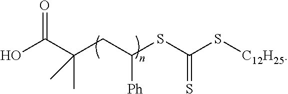

In some cases, the chain transfer agent comprises a mixture of (4-cyano-4-(phenyl-carbonothioylthio)pentanoic acid), (4-cyano-4-[(ethylsulfanylthiocarbonyl)sulfanyl]pentanoic acid), (4-cyano-4-[(dodecyl sulfanylthiocarbonyl)sulfanyl]pentanoic acid), and/or (2-cyano-2-propyl dodecyl trithiocarbonate).

In some cases, the method comprises admixing a plurality of a first monomer precursor and the second monomer under conditions to polymerize the first monomer precursor and the second monomer to form a copolymer precursor, optionally in the presence of a polymerization initiator, a chain transfer agent, or both; and reacting the copolymer precursor with chloroquine or a chloroquine analog to append the chloroquine or the chloroquine analog to the copolymer precursor to form the copolymer. In some cases, the first monomer precursor comprises an alkyne moiety, and the chloroquine or chloroquine analog comprises an azide moiety (or vice versa), and the reacting comprises formation of a triazolyl between the alkyne and the azide to append the chloroquine or the chloroquine analog.

In general, copolymers as described herein can be synthesized according to Scheme 1.

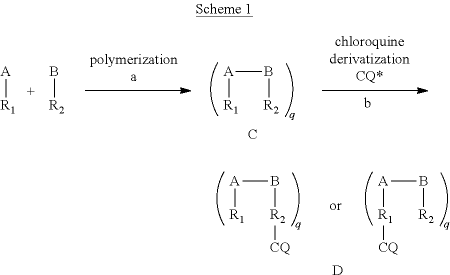

Copolymers having structure D can be synthesized using the procedure shown in Scheme 1. Copolymerization of monomers A and B under appropriate reaction conditions a form a copolymer (structure C) having a number of repeats q. For example, if A and B are both ethylene derivatives, the polymerization can be carried out under RAFT (reversible addition-fragmentation chain transfer) conditions, e.g., use of a chain transfer agent (CTA) and a radical initiator such as AIBN in a solvent or mixture of solvents (e.g., methanol, 1,4-dioxane, and/or DMSO) at elevated temperature (e.g., at 70° C.). Copolymer C can then be reacted with chloroquine or a chloroquine analog CQ* under appropriate reaction conditions b to yield a chloroquine-containing copolymer D. The conditions for the chloroquine derivatization can be selected based on the nature of monomers A and B. For example, in Scheme 1 above, monomers A and B are shown bearing side chains R₁ and R₂, respectively. These side chains R₁ and R₂ can be selected to react with CQ* to yield a chloroquine-containing copolymer D. For example, one of R₁ and R₂ can comprise an azide moiety, and CQ* can comprise an alkyne moiety which can undergo a “click” reaction to modify the copolymer C with chloroquine, yielding a modified copolymer D. Alternatively, one of R₁ and R₂ can be an alkyne moiety, and CQ* can comprise an azide moiety.

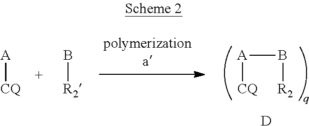

Copolymers having structure D can be also synthesized using the procedure shown in Scheme 2. Copolymerization of monomers A and B under appropriate reaction conditions forms a copolymer (structure D) having a number of repeats q. For example, if A and B are both ethylene derivatives, the polymerization can be carried out under RAFT (reversible addition-fragmentation chain transfer) conditions, e.g., use of a chain transfer agent (CTA) and a radical initiator such as AIBN in a solvent or mixture of solvents (e.g., methanol, 1,4-dioxane, and/or DMSO) at elevated temperature (e.g., at 70° C.). In Scheme 2 above, monomer A is derivatized with chloroquine before the polymerization reaction, in order to yield a chloroquine-containing copolymer D upon reaction with monomer B. Monomer B can comprise a radical R_{2′} which allows for optional further derivatization of the copolymer.

Additional synthetic procedures for preparing the compounds disclosed herein can be found in the Examples section.

### Methods of Use

The copolymers disclosed herein can be used in many different therapeutic applications, such as treatment of IBD or cancer. The co-polymers can be administered directly to a patient in need. The co-polymers may also be formed into beads, microparticles and/or nanoparticles before being administered to a patient. In some cases, the administering comprises oral administration.

Disclosed herein are methods of treating inflammatory bowel disease (IBD) comprising administering to a patient in need thereof a therapeutically effective amount of a copolymer described herein, or a bead, microparticle, or nanoparticle described herein. In some cases, IBD comprises ulcerative colitis, Crohn's disease, irritable bowel syndrome, amebic colitis, acute self-limiting colitis, or colitis. In some cases, IBD comprises Crohn's disease. In some cases, IBD comprises irritable bowel syndrome. In some cases, IBD comprises ulcerative colitis, amebic colitis, acute self-limiting colitis, or colitis. In some cases, IBD comprises ulcerative colitis. In some cases, IBD comprises amebic colitis. In some cases, IBD comprises acute self-limiting colitis. In some cases, IBD comprises colitis.

Also disclosed herein are methods of treating cancer comprising administering to a patient in need thereof a therapeutically effective amount of a copolymer described herein, or a bead, microparticle, or nanoparticle described herein. In some cases, the patient suffers from colon cancer. In some cases, the methods of treating cancer comprise administering an anti-cancer agents in addition to the co-polymers, beads, microparticles, and nanoparticles of the present disclosure. In some cases, co-polymers, beads, microparticles, and nanoparticles of the present disclosure can be administered with an anti-cancer agent. In some cases, the anti-cancer agent comprises small molecules, biologics (peptides, DNA, RNA, proteins, antibodies, and antibody fragments), and combinations thereof. In some cases, the anti-cancer agent is loaded or encapsulated within the co-polymers and/or beads, microparticles, and nanoparticles of the present disclosure.

The co-polymers and beads, microparticles, and nanoparticles of the present disclosure may be administered by any suitable method. In some cases, the beads, microparticles, and nanoparticles are administered orally, intravenously, rectally, subcutaneously, or intramuscularly. In some cases, the route of administration is orally or rectally. The co-polymers and beads, microparticles, and nanoparticles of the present disclosure can be designed so that they may be delivered orally, and due to their size (molecular weight), the co-polymers will not be taken up systemically to any large extend and are primarily localized within the gastrointestinal tract.

### EXAMPLES

The following examples are provided for illustration and are not intended to limit the scope of the disclosure.

### Synthetic Procedures for Copolymers

General Experimental Procedures. All reagents and solvents were obtained from commercial sources and used without additional purification.

### Example 1—Synthesis of Chloroquine Copolymers

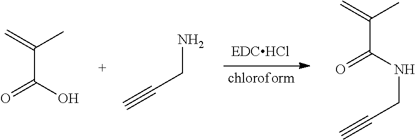

### Synthesis of N-propargyl Methacrylamide (PPMA)

Methacrylic acid (20 mmol, 1721 mg, 1687 μL) and 1-(3-dimethylaminopropyl)-3-ethylcarbodiimide hydrochloride (EDC-HCl, 30 mmol, 5751 mg) were dissolved in anhydrous chloroform (60 mL) and stirred at room temperature for 30 min. The mixture was cooled down in ice bath followed by addition of propargyl amine (13.33 mmol, 734 mg, 854 μL) dissolved in chloroform (15 mL). After stirring overnight, the resulting product was concentrated and purified by column chromatography with dichloromethane:methanol=10:1 as eluent.

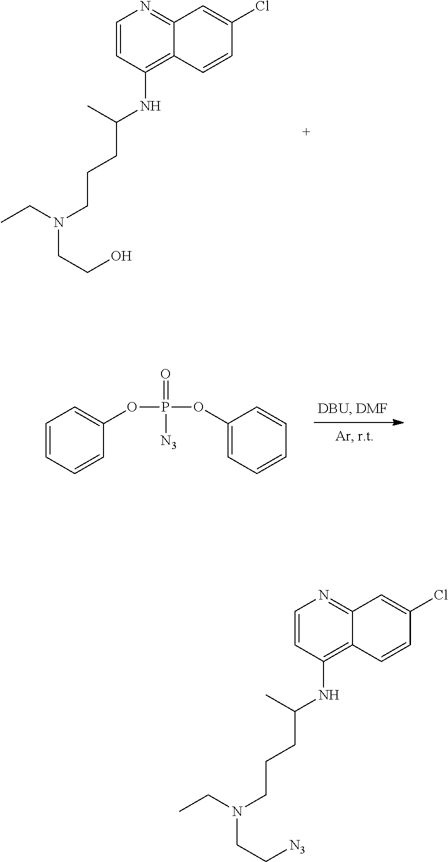 [SMILES: CCN(CCO)CCCC(C)Nc1ccnc2cc(Cl)ccc12 ; [N-]=[N+]=NP(=O)(Oc1ccccc1)Oc1ccccc1 ; CCN(CCCC(C)Nc1ccnc2cc(Cl)ccc12)CCN=[N+]=[N-]]

### Synthesis of CQ-N₃

HCQ (360 mg, 1.07 mmol) and diphenylphosphoryl azide (338.5 mg, 1.23 mmol, 265 μL) were dissolved in dimethylformamide under argon. Then 1,8-Diazabicyclo(5.4.0)undec-7-ene (DBU, 190 mg, 1.23 mmol, 186 μL) was added and stirred for 48 h. The resulting product was diluted by dichloromethane (50 mL) and washed by deionized water (50 mL) twice and brine (50 mL). The organic phase was dried through sodium sulfate and evaporated by rotavap. The final product was purified by column chromatography with dichloromethane:methanol=10:1 as eluent.

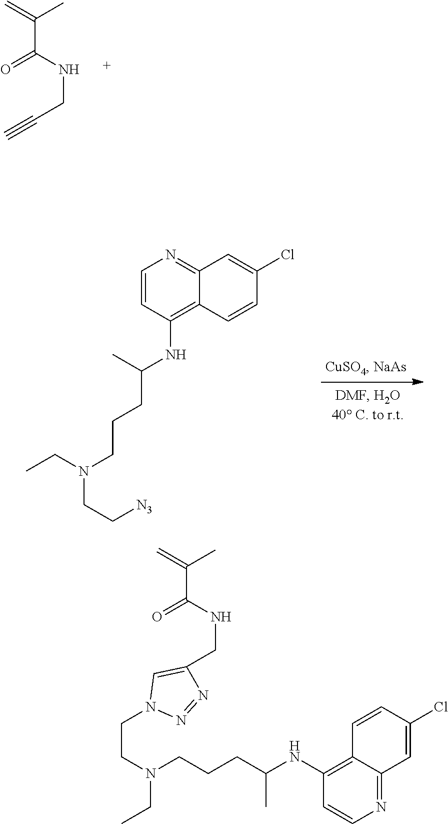

### Synthesis of MA-tCQ

CQ-N₃ (343 mg, 0.95 mmol) and PPMA (185 mg, 1.5 mmol) were dissolved in DMF (2.5 mL). CuSO₄ (8 mg, 0.05 mmol) was dissolved in deionized water (0.4 mL) and added into the DMF solution. After the reaction mixture was degassed for 30 min, sodium ascorbate (40 mg, 0.2 mmol) dissolved in deionized water (0.4 mL) was added to the above reaction mixture under N₂. The reaction was taken at 40° C. for 2 h and at room temperature overnight with stirring. The resulting mixture was diluted by DCM (50 mL) and washed by EDTA aqueous solution (50 mM, 50 mL) twice, water (50 mL) and brine (50 mL). The organic phase was dried through sodium sulfate and evaporated by rotavap. The final product was purified by column chromatography with dichloromethane:methanol=10:1 as eluent.

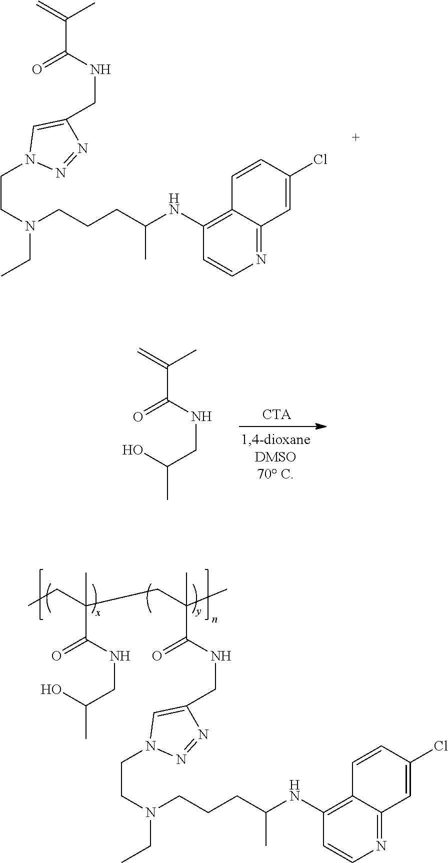

### Synthesis of NpCQ

Polymers with different CQ content were obtained by changing the monomer ratio of MA-tCQ to HPMA in the reagents, which was similar to the preparation of pCQ. Typically, MA-tCQ (70 mg, 0.15 mmol), HPMA (103 mg, 0.72 mmol), CTA and AIBN were dissolved in methanol:1,4-dioxane=1:1 (1 mL) and purged by nitrogen for 30 min. After stirred at 70° C. overnight, the polymer was precipitated out by adding the mixture to cold diethyl ether under vigorous stirring. The precipitates were collected and re-dissolved in methanol. The precipitation step was repeated twice and the polymers were dialyzed against water (MWCO: 3,500) for 2 days. The NpCQ polymers were obtained by lyophilization.

### Example 2—Synthesis of Clickable HPMA-Containing Polymers

Investigation of chain transfer agent for polymerization of Poly(HPMA-co-PPMA) (pHP)

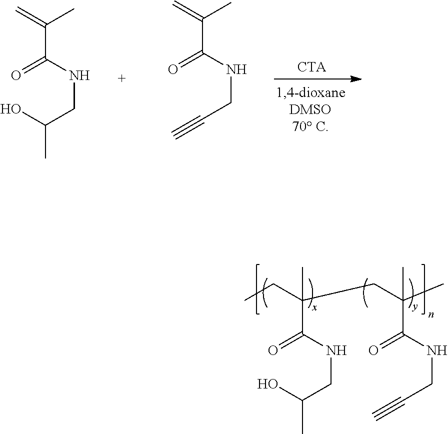

### Chain-Transfer Agent Synthesis of pHP

Reversible addition-fragmentation chain transfer (RAFT) polymerization was applied to synthesis of clickable HPMA and PPMA copolymers. Due to different monomer compatibility of RAFT agents, four CTAs were tested in the polymerization of pHP with PPMA content as 20% (mol %). One dithiobenzoate and three trithiocarbonates CTAs were chosen due to their reported compatibility to methacrylamides. The molar ratio of HPMA/PPMA/CTA/initiator was fixed at 80/20/1/0.25. HPMA, PPMA, AIBN and the CTAs were dissolved in 1,4-dioxane/DMSO (1/1, v/v, 100 mg/mL) and transferred to prescored ampoules. The ampoules were flame sealed after purged by nitrogen for 30 min. The polymerization was conducted at 70° C. for 16 h. The polymers were precipitated in cold diethyl ether for three times and dried under vacuum. The composition of each polymer was analyzed by ¹H NMR. The molecular weight of the polymers were tested by GPC as water soluble cationic polymers. Characterization data of pPPMA polymer prepared with different chain transfer agents is presented in Table 1, below.

TABLE 1

| | | | | | PPMA in |
|---|---|---|---|---|---|
| | [HPMA]₀/ | | | | feed/ |
| | [PPMA]₀/ | Mₜₕ | M_{W} | | in polymer |
| CTA | [CTA]₀/[I]₀ | (kDa) | (kDa) | PDI | (mol %) |
| 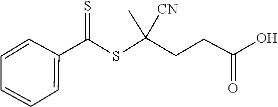 [SMILES: CC(C#N)(CCC(=O)O)SC(=S)c1ccccc1] | 80/20/1/0.25 | 13.9 | — | — | 20/- |
| 4-cyano-4-(phenyl-carbonothioylthio) | | | | | |
| pentanoic acid | | | | | |
| 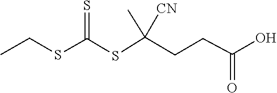 | 80/20/1/0.25 | 13.9 | 7.5 | 1.1 | 20/19.7 |
| 4-cyano-4-[(ethylsulfanylthiocarbonyl) | | | | | |
| sulfanyl]pentanoic acid | | | | | |
| 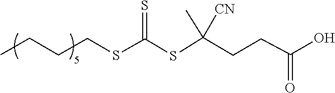 | 80/20/1/0.25 | 13.9 | 9.3 | 1.2 | 20/20.0 |
| 4-cyano-4-[(dodecyl sulfanylthiocarbonyl) | | | | | |
| sulfanyl]pentanoic acid | | | | | |
| 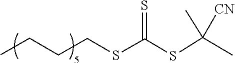 | 80/20/1/0.25 | 13.9 | 13.6 | 1.2 | 20/19.7 |
| 2-cyano-2-propyl dodecyl | | | | | |
| trithiocarbonate | | | | | |

### Synthesis of pHP Copolymer

The pHP copolymer was prepared using 2-cyano-2-propyl dodecyl trithiocarbonate as CTA agent and AIBN as initiator. The copolymers were synthesized with arrange of molecular weights 7-56 kDa and different PPMA contents (Table 2). The procedure was the same as described above.

TABLE 2

| | | | | | PPMA in feed/ |
|---|---|---|---|---|---|
| | [HPMA]₀/[PPMA]₀/ | Mₜₕ | M_{w} | | in polymer |
| Sample | [CTA]₀/[I]₀ | (kDa) | (kDa) | PDI | (mol %) |
| pHP0 | 100/0/1/0.25 | 14.3 | 14.8 | 1.1 | 0/0 |
| pHP5 | 95/5/1/0.25 | 14.2 | 16.7 | 1.2 | 5/4.3 |
| pHP10 | 90/10/1/0.25 | 14.1 | 14.3 | 1.2 | 10/9.9 |
| pHP20 | 80/20/1/0.25 | 13.9 | 13.6 | 1.2 | 20/19.7 |
| pHP40 | 60/40/1/0.25 | 13.5 | 11.3 | 1.4 | 40/24.4 |
| pHP20L | 80/20/2/0.5 | 7.0 | 7.8 | 1.2 | 20/16.5 |
| pHP20M | 80/20/0.5/0.125 | 27.8 | 23.8 | 1.4 | 20/19.5 |
| pHP20H | 80/20/0.25/0.0625 | 55.6 | 38.1 | 1.4 | 20/20.6 |

### Click CQ-N3 to pHP

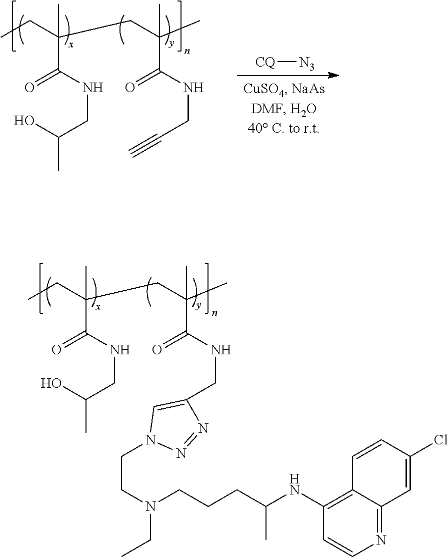

To conduct the click reaction, pHP, CQ-N3 (1.1 equiv. of alkyne amount in pHP) and CuSO₄ (0.1 equiv. of CQ-N3) were dissolved in water containing 10% DMF under nitrogen in a Schlenk tube. The reaction mixture was purged with nitrogen for 30 min. NaAs (0.4 equiv. of CQ-N₃) was added before the tube was submerged in 40° C. oil bath. The reaction mixture was stirred at 40° C. for 2 h and cooled down to room temperature overnight. The resulting product was washed by 50 mM EDTA aqueous solution twice and water once to remove the copper ion, followed by dialysis against water (MWCO: 3,500) for 3 days. The final product was obtained by lyophilization. The molecular weight was obtained by GPC and CQ content was calculated by ¹H NMR.

### Synthesis of pHPte Polymers by RAFT Polymerization

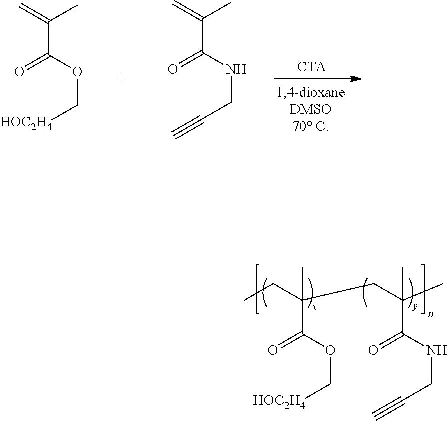

A copolymer of alkyne-containing poly(2-hydroxypropyl methacrylate) was prepared as described above using 4-cyano-4-[(ethylsulfanylthiocarbonyl)sulfanyl]pentanoic acid as the CTA (Table 3, below).

TABLE 3

| | | | | | PPMA in feed/ |
|---|---|---|---|---|---|
| | [HPMAte]₀/[PPMA]₀/ | Mₜₕ | M_{w} | | in polymer |
| Sample | [CTA]₀/[I]₀ | (kDa) | (kDa) | PDI | (mol %) |
| pHPte0 | 100/0/1/0.25 | 14.4 | 17.6 | 1.1 | 0/0 |
| pHPte5 | 95/5/1/0.25 | 14.3 | 19.2 | 1.1 | 5.0/4.8 |
| pHPte10 | 90/10/1/0.25 | 14.2 | 19.9 | 1.1 | 10.0/8.3 |
| pHPte20 | 80/20/1/0.25 | 14.0 | 20.5 | 1.1 | 20.0/15.3 |
| pHPte40 | 60/40/1/0.25 | 13.6 | 16.9 | 1.2 | 40.0/28.0 |
| pHPte20L | 80/20/2/0.5 | 7.2 | 9.0 | 1.1 | 20.0/16.7 |
| pHPte20M | 80/20/0.5/0.125 | 28.0 | 32.9 | 1.2 | 20.0/16.7 |
| pHPte20H | 80/20/0.25/0.0625 | 55.9 | 51.2 | 1.1 | 20.0/15.3 |

### Biological Assay Data

### Example 3—Rodent Model of Colitis

One of the major challenges in testing novel oral drug delivery systems in IBD is mimicking the GI environment in vitro, particularly because of the diversity in the pH as well as presence of various enzymes. Consequently, animal models form an indispensable part of the IBD research. One of the most commonly used animal models of colitis is a chemical injury model using dextran sodium sulfate (DSS). The mechanism by which DSS induces colitis is not entirely clear. However, it is postulated that the inflammation is likely due to the damage caused to the epithelial monolayer allowing dissemination of luminal colonic contents into the underlying tissue. The DSS model is reproducible and the severity of the colitis can be controlled by adjusting the dose and frequency of DSS administration. One of the main advantages of the DSS-induced colitis is its histological similarity to human colitis. However, recent reports have challenged the biorelevance of the DSS model from a pathological perspective. Unlike human IBD, the DSS model lacks a clearly defined involvement of enteric bacterial flora. Another concern with the DSS model is that following ingestion, DSS forms nanosized vesicles with fatty acids in the colon and is deposited onto the colonic epithelium, essentially forming a physical barrier which can interfere in the interaction between therapeutics and drug delivery systems and the intestinal tissue. Moreover, unlike human IBD, the DSS model does not verify a clearly defined involvement of enteric bacterial flora [40]. In addition, interference of DSS with RT-PCR is an established concern.

To overcome the limitations of the DSS-colitis model of murine colitis and also to account for the involvement for the microbial-induced inflammatory signaling, as in IBD, we used the murine model of infectious colitis, which is frequently used for modeling the IBD in mouse. Notably, the C. rodentium-murine-specific bacteria that is closely related to important human pathogen E. soli. It attaches to the apical side of the colon epithelium and invades the colon wall, thus eliciting an immune response. In addition to being well characterized for understanding host responses to enteric bacteria, it produces robust colitis characterized by elongation of crypts, immune cell infiltration, and goblet cell depletion. Unlike the DSS colitis, which requires continuous administration of the DSS, the C. rodentium model uses a single gavage of the bacteria, and thus reduces the stress on the mice and minimizes the likelihood of interference with the tested drug delivery systems.

Hydroxychloroquine (HCQ) sulfate (98%), triethylamine, DMSO-d₆ (99.8%) and chloroform-d (99.8%) were obtained from Acros Organics (Fisher Scientific). Methanol, acetonitrile (HPLC grade) were purchased from Fisher Scientific. pHP-CQ (pCQ) with 16.7 mol % of CQ and weight average molecular weight 60 kDa was synthesized and characterized as described above.

Male C57BL/6 mice (6 weeks old, 18-20 g) were obtained from Charles River Laboratories and used for all in vivo studies. All animal experiments were conducted according to the protocol approved by the University of Nebraska Medical Center Institutional Animal Care and Use Committee. C. rodentium was used to induce colitis in mice. Bacterial glycerol stock was streaked onto LB agar plates and the bacterial colonies were grown overnight at 37° C. A single colony was inoculated into 5 ml of 2% LB broth and incubated at 37° C., 280 rpm overnight to obtain a saturated primary culture. On the day of the experiment (Day 0), 2 ml of the primary culture was added into 300 ml of LB broth and the inoculum was placed on a shaker at 37° C. at 280 rpm for 6 hours. Bacterial optical density was measured at 600 nm. Bacteria were diluted with LB broth and delivered to mice (n=15) via oral gavage in a 100 μL volume containing 5×108 colony forming units (CFU). Healthy control group (n=5) was orally administered 100 μL of LB broth.

### Statistics

The Mann-Whitney test was used for statistical analysis of mean differences between treatment groups for biodistribution studies. One-way ANOVA followed by Tukey multiple comparison test was used for statistical analysis of mean differences among multiple groups. A value of p<0.05 was considered statistically significant. All statistical analysis was performed using Graphpad Prism v5 (ns=not significant, *=p≤0.05, **=p≤0.01, ***=p≤0.001).

### Example 4—Cytokine Analysis

At least five mice were sacrificed at each time-point (day 1, 10 and 14 after oral gavage of the C. rodentium). The colons were excised, cleaned, and their weight and length recorded. The entire colon was frozen and stored at −80° C. The colon was homogenized in 1.5 ml RIPA buffer using TissueLyserII (Qiagen) and the homogenates centrifuged at 15,000×g for 15 min at 4° C. The supernatant was analyzed for cytokine levels using ProcartaPlex™ Multiplex Immunoassay (Invitrogen) following the manufacturer's protocol. Briefly, magnetic beads coated with different cytokine antibodies were added at the required dilution to 96-well plate. Supernatants from the tissue homogenates and supplied fluorescent standards were added to the respective wells. The cytokines were detected by adding a detection antibody followed by addition of Streptavidin-PE. The beads were read on Luminex™ LX 200 Analyzer. The data obtained were analyzed using ProcartaPlex™ Analyst software to obtain cytokine concentrations.

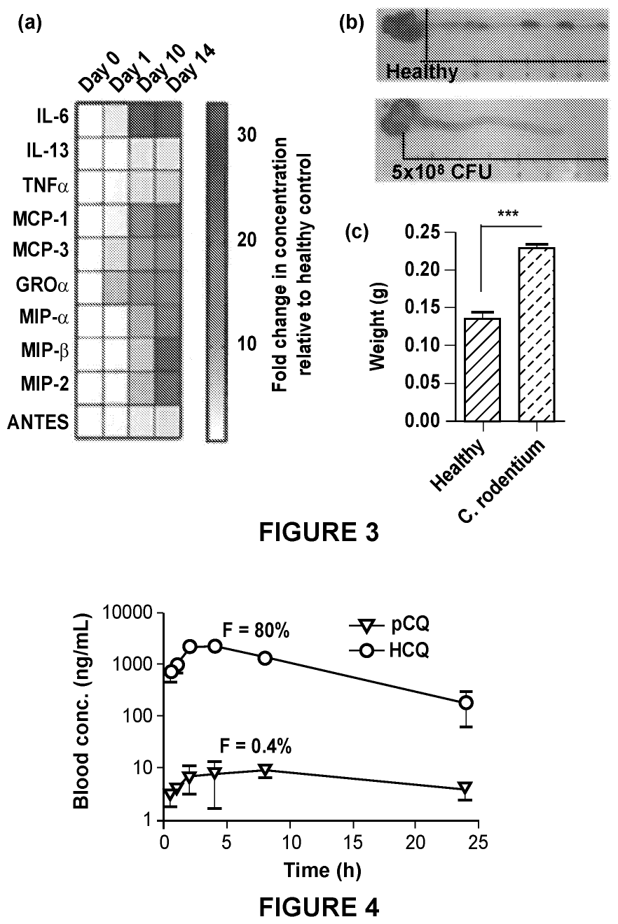

It was first verified that the C. rodentium model exhibited expected pathobiology similar to the human IBD. As expected, significant upregulation in the expression of pro-inflammatory cytokines was observed over 14 days after single oral dose of the bacteria (FIG. 3a). IL-6 was the most upregulated cytokine with almost 30-times higher peak levels than healthy controls. Other pro-inflammatory cytokines showed a similar trend. Further, the colons from mice infected with the bacteria showed significant reduction in length (FIG. 3b) and a contrasting increase in its weight (FIG. 3c) as a result of the inflammation and edema. Histologically, colons of the infected mice exhibited inflammation-associated epithelial changes evidenced by crypt elongation, crypt fall out, presence of apoptotic cells, and significant inflammatory cell infiltration. Distal colon showed more pronounced changes than the proximal colon, which was consistent with previous findings suggesting that C. rodentium preferentially colonizes the distal colon. The observed parameters correlate well with the chronic inflammatory changes, which occur during the development of human IBD. Overall, these findings supported the suitability of this animal model considering that it presented a well-defined chronic inflammatory condition of the mucosa with significant similarities to the IBD.

### Example 5—Pharmacokinetics and Biodistribution

The pharmacokinetics and biodistribution of single dose pCQ was evaluated in a mouse model of IBD. Colitis was induced as described above and on day 14, mice were given a single dose of HCQ or pCQ in 200 μl of deionized water via oral gavage equivalent to 30 mg/kg HCQ. Blood was collected from the submandibular vein at 0.5, 1, 2, 4, 8, and 24 h post-administration. The animals were sacrificed, and organs harvested at 0.5, 2, 8, and 24 h post-administration and stored at −80° C. until further use. The mice were randomized to selected sampling times so that three blood samples and one terminal tissue collection were obtained from each.

Blood, tissue and fecal samples were processed by two methods to determine free HCQ and total HCQ. For the first method, HCQ was isolated by a simple extraction from the organs as described in Murry et al., J. Chrom. B., (2018) 1072, 320-327. The second method utilized base hydrolysis to release HCQ covalently bound to pCQ and subsequent HCQ extraction using solid phase extraction (SPE). Tissues and feces were homogenized in water prior to loading to the SPE cartridge. The calibration and quality control samples were separately prepared for HCQ and pCQ by spiking 10 μl of appropriate calibration stock of HCQ and pCQ, in 100 μl of blank biomatrix, and 10 μl of internal standard solution (1.0 μg/ml) added. For the study, 25 μl of blood and 100 μl of tissue homogenate were used. 400 μl of 1 M NaOH, 600 μl water and 100 μl methanol were added and the samples were incubated at 50° C. for 1 h to hydrolyze HCQ from the pCQ. Subsequently, 400 μ12% formic acid was added and the samples were vortexed for 30 s and centrifuged at 1301×g for 10 min. The SPE was carried out using Oasis HLB 3cc, 60 mg extraction Cartridge (Waters, Inc). Cartridges were pre-conditioned with 1 ml acetonitrile followed by 1 ml water. Samples were loaded to the cartridges and washed with 2 ml of aqueous 15% methanol and dried at high vacuum for 15 min. Analytes were eluted with 2 ml of acetonitrile. The eluents were collected in glass tubes and dried under nitrogen in water bath at 50° C. The dry residues were reconstituted in 400 μ1 0.1% formic acid:methanol mixture (60:40) and centrifuged at 13000×g and 10 μl supernatant was injected into the HPLC. The LC-MS/MS conditions were as previously described. (Murry et al., J. Chrom. B., (2018) 1072, 320-327.) The assay was linear over the range of 1 to 2000 ng/mL.

The pharmacokinetic parameters of HCQ and pCQ in blood and tissues were calculated using non-compartmental analysis with Phoenix WinNonlin 6.3 (Pharsight Corporation). The maximum concentration (Cₘₐₓ) and time to Cₘₐₓ (Tₘₐₓ) were obtained from the visual inspection of the concentration-time plot. The area under the curve (AUC_{0-∞}) was estimated using the linear trapezoidal method from 0-tₗₐₛₜ and extrapolation from tₗₐₛₜ to infinity based on the observed concentration at the last time point divided by the terminal elimination rate constant (λ). The elimination half-life (t_{1/2}) was calculated as 0.693/k. Apparent clearance (CI/F) and the apparent volume of distribution of the elimination phase (V_{d}/F) were calculated as dose/AUC_{0-∞} and dose/k*AUC_{0-∞}, respectively. The mean residence time (MRT) was calculated as AUMC_{0-∞}/AUC_{0-∞}. Mean tissue concentrations were calculated and expressed as ng/g tissue. The absolute bioavailability was calculated as:

$${F}_{abs} = \frac{{AUC}_{po} \times {D}_{iv}}{{AUC}_{iv} \times {D}_{po}} ;$$

where AUCₚₒ and AUCᵢᵥ are the areas under the curve after oral (po) and i.v. administration and Dᵢᵥ and Dₚₒ are the corresponding doses.

The pCQ contains HCQ attached by a potentially hydrolyzable ester linker and it was thus important to distinguish between polymer-conjugated HCQ and free HCQ released from pCQ. This was achieved by using two LC-MS/MS analytical methods. First, HCQ was quantified using a simple extraction from blood and tissues to determine the amount of HCQ that was released from pCQ by hydrolysis in the GI tract, blood, or liver. In the second method, an alkaline hydrolysis step was included that was optimized to fully hydrolyze the ester linker between HCQ and the polymer, thus providing information on the total (polymer bound+hydrolyzed) HCQ. The difference in the HCQ amount obtained from the two methods was used to calculate the percent of HCQ in the tissues that remained bound to the polymer. The HCQ amount quantified by both methods was similar in blood and tissues from animals treated with HCQ.

The blood concentration vs. time profile for the HCQ and pCQ after oral administration is shown in FIG. 4 and the blood pharmacokinetic data in Table 4. The pharmacokinetic parameters of pCQ were determined from the total HCQ content in the blood and thus represent a combined pharmacokinetics of polymer-bound and hydrolyzed HCQ.

TABLE 4

| Pharmacokinetic | | |
|---|---|---|
| parameter | HCQ | pCQ |
| Cₘₐₓ (ng/ml) | 2,343 ± 46 | 12.2 ± 1.7 |
| t_{1/2} (h) | 5.6 ± 1.9 | 11.7 ± 3.6 |
| tₘₐₓ (h) | 3.3 ± 1.2 | 4.7 ± 3.1 |
| AUC₀₋ₗₐₛₜ (h × ng/ml) | 26,446 ± 507 | 161 ± 8 |
| AUC_{0-∞} (h × ng/ml) | 28,182 ± 1476 | 231 ± 49 |
| Cl/F (L/h/kg) | 1.1 ± 0.1 | 134 ± 27 |
| MRT (h) | 6.8 ± 0.6 | 9.9 ± 0.7 |

The blood concentration vs. time profile for the HCQ and pCQ after oral administration is shown in FIG. 4. The PK parameters of pCQ were determined from the total HCQ content in the blood and thus represent a combined PK of polymer-bound and hydrolyzed HCQ. The drug reached a maximum concentration in blood (Cₘₐₓ) of 2,342.6±46.3 and 12.2±1.7 ng/mL for HCQ and pCQ treatment, respectively. The values of area under curve (AUC_{0-∞}) were determined as 28,182.4±1,475.8 and 231.3±48.8 hr×ng/mL for HCQ and pCQ treatment, respectively. In comparison to HCQ, the pCQ formulation exhibited a significant reduction of Cₘₐₓ (˜192-fold) and AUC_{0-∞} (˜122 fold) indicating that modifying HCQ into pCQ dramatically reduced its absorption. The absolute bioavailability (oral to i.v.), was found to be 0.4% for pCQ compared to 80% for HCQ indicating that the pCQ formulation substantially reduced HCQ bioavailability and maintained drug in the GI tract. One pharmacokinetic disadvantage of chloroquine and its metabolites is their exceptionally long residence time in the blood leading to severe side effects. In pCQ treatment group, HCQ levels were substantially lower in the systemic circulation, suggesting that prolonged exposure to HCQ and metabolites will not be a major systemic toxicant. This is an important finding for a small molecule drug like chloroquine which has high systemic bioavailability resulting in high non-specific tissue exposure. Reduction in systemic absorption and bioavailability is important for local therapy and reduction of systemic toxicities and the pCQ formulation resulted in a very different blood PK profile compared to HCQ.

### Colon and Liver Distribution

There have been numerous reports indicating that conjugating small molecule drugs to polymers can change their pharmacokinetics and pre-dispose them to preferential accumulation in specific tissues. An analysis was carried out of how differences in blood pharmacokinetics affect relative distribution of pCQ and HCQ to the colon and liver following oral administration in the C. rodentium colitis.

TABLE 5

| Pharmacokinetic | Liver | | Colon | |
|---|---|---|---|---|
| parameter | HCQ | pCQ | HCQ | pCQ |
| Cₘₐₓ (ng/ml) | 12,807 ± 3703 | 220.2 ± 102.7 | 10,304 ± 746 | 7,121 ± 2,984 |
| tₘₐₓ (h) | 2.0 ± 0.0 | 0.5 ± 0.0 | 8.0 ± 0.0 | 8.0 ± 0.0 |
| AUC₀₋ₗₐₛₜ (h × ng/ml) | 167,944 ± 19,302 | 1,547 ± 100 | 166,377 ± 14,873 | 93,088 ± 34,403 |
| AUC_{0-∞} (h × ng/ml) | 175,852 ± 18,362 | 1,659 ± 78 | 208,917 ± 55,806 | 94,515 ± 35,363 |

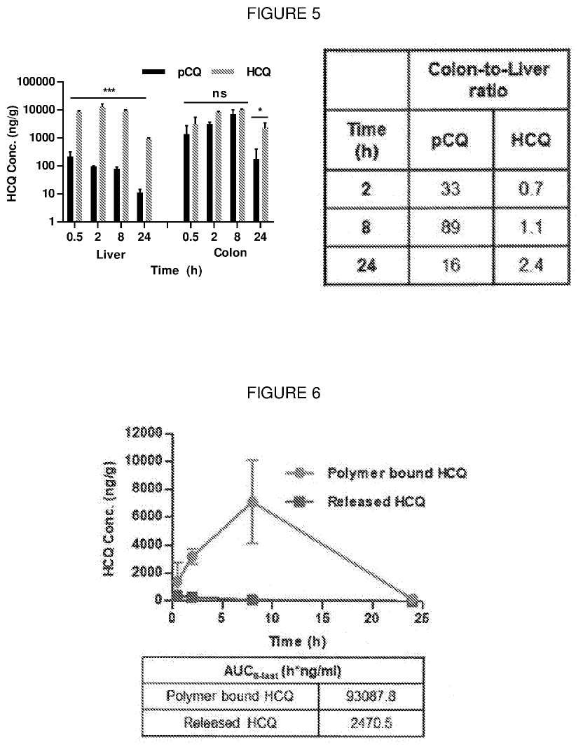

The colon and liver PK and distribution results are shown in Table 5 and FIG. 5. HCQ and pCQ reached a Cₘₐₓ of HCQ in colon of 10,304±746 and 7,121±2,984 ng/ml, respectively. The colon AUC_{0-∞} was 208,917±55,806 for HCQ and 94,515±35,363 hr×ng/mL for pCQ treatment. HCQ appeared to show higher colon concentrations than pCQ probably due to faster transit time but the difference did not reach statistical significance. Both HCQ and pCQ showed increasing accumulation in the colon from the time of administration until at least 8 h, with subsequent decline by 24 h (FIG. 5). Both pCQ and HCQ showed similar colon PK behavior. The Tₘₐₓ for HCQ and pCQ occurred at 8 h. However, major differences were observed in the hepatic PK parameters of pCQ and HCQ. As expected from the very low bioavailability, pCQ had much lower hepatic accumulation than HCQ with the liver Cₘₐₓ for pCQ 58-times lower than the HCQ and ˜110-times lower AUC₀₋ₗₐₛₜ compared to HCQ. It was noteworthy that pCQ concentrations in the liver declined from the first measured time point and were at all times lower than the liver levels of HCQ. These PK differences contributed to the preferential localization of pCQ in the colon as suggested by the calculated colon-to-liver ratios in FIG. 5. The pCQ colon:liver ratio was higher at all measure time points compared to HCQ treatment. Fecal pCQ concentrations were higher than HCQ levels. These observations reinforce the applicability of pCQ as a local colonic treatment.

Having established the local colon accumulation of pCQ, an analysis of pCQ hydrolysis in the GI tract and the extent of release of free HCQ was undertaken. An analysis of the content of polymer-bound HCQ and the extent of pCQ hydrolysis using the two different sample preparation methods described above was carried out. As shown in FIG. 6, the vast majority of the HCQ was polymer-bound until at least 8 h post-administration. The released (i.e., free) HCQ levels in the colon decreased, whereas the polymer-bound HCQ levels increased over time. While the released HCQ concentrations were highest in the colon at 1.5 h, the polymer-bound HCQ achieved maximum concentrations at 8 h. Calculating the hydrolyzed fraction at 8 h, only 1.2% of free HCQ was present in the colon tissue. This indicated that the hydrolyzed HCQ was systemically absorbed while the polymer-bound HCQ had a higher transit time to localize in the colon before clearance at 24 h. Estimate of the colon AUC₀₋ₗₐₛₜ showed about 37-fold difference between the polymer-bound HCQ and HCQ hydrolyzed from pCQ, suggesting that most of the therapeutic effects described below resulted from the activity of pCQ and not released HCQ.

### HCQ and pCQ Metabolism

To address tissue accumulation and subsequent metabolism of HCQ and pCQ, the concentrations of HCQ metabolites in colon and liver were measured at serial time points following oral administration. HCQ is metabolized in the liver by dealkylation into three major metabolites: desethylchloroquine (DCQ), bisdesethylchloroquine (BDCQ) and desethylhydroxychloroquine (DHCQ. It was previously shown that DCQ has similar antimalarial activity as HCQ. All the N-dealkylated metabolites have been implicated in heart failure and retinopathy, with BDCQ being more cardiotoxic than HCQ [44]. Importantly for chronic use in IBD, HCQ and its metabolites have extremely long biological half-lives and thus their monitoring is important.

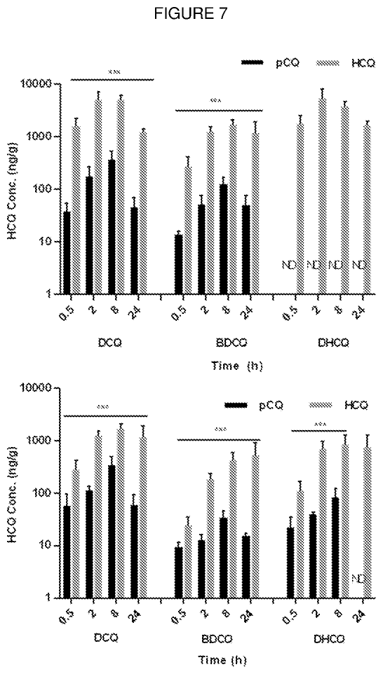

As expected, pCQ significantly decreased the extent and rate of HCQ metabolism due to the covalent bond formed between the polymer and the hydroxyl in HCQ. The metabolite concentration results in liver and colon are shown in FIG. 7. In the liver (FIG. 7a), which is the main organ for HCQ metabolism, both DCQ and BDCQ concentrations were 10-100-fold higher in the HCQ treated group as compared to the pCQ group. Both DCQ and BDCQ liver concentrations peaked at 8 h. DHCQ liver levels were undetectable in the pCQ group. Analysis of blood metabolite concentrations revealed similar trend as most metabolites were either undetectable or significantly lower in the pCQ group as a result of the very low bioavailability.

Data in FIG. 7b suggest that HCQ metabolism occurs in the colon. In agreement with the liver metabolism findings, metabolite concentrations were measured to be about 10-100-fold lower in the pCQ group than in the HCQ group. Calculating the percent of metabolites in colon at Cₘₐₓ (8 h), pCQ was metabolized to a lower extent than HCQ. While the major metabolite DCQ accounted for 16.5% in the HCQ group, only 4% of pCQ was metabolized to DCQ in the colon. BDCQ (4% of HCQ vs. 0.4% of pCQ) and DHCQ (8% of HCQ vs. 1% of pCQ) showed similar differences. This finding, coupled with the observation of elevated fecal pCQ concentrations, further support pCQ localization in the colon as opposed to systemic absorption. The covalent conjugation of HCQ in pCQ not only reduced its oral absorption due to its macromolecular nature but it also decreased accessibility to metabolic enzymes, thus preventing generation of toxic HCQ metabolites. Such a low systemic exposure to HCQ and its metabolites may result in reduction of adverse systemic side effects commonly observed with HCQ.

### Example 6—Therapeutic Efficacy

Colitis was induced as stated above and mice were randomly assigned to healthy, HCQ, and pCQ treatment groups (n=5) and untreated group (n=8). Starting day 1, the mice received oral gavage of either HCQ or pCQ every other day (30 mg/kg HCQ equivalent in 200 μl sterile deionized water). Untreated and healthy controls were administered 200 μl sterile deionized water. On day 14, the mice were sacrificed and the colons were harvested. The colon was opened longitudinally, cleaned of fecal matter, and excised into two parts along the length, which were stored accordingly for determination of cytokine mRNA levels by RT-PCR and histological analysis.

The longitudinally opened colons were rolled into a Swiss roll from distal to proximal end. The rolls were fixed for 24 h in 4% paraformaldehyde, embedded in paraffin, sectioned and stained with hematoxylin and eosin (H&E). The stained sections were evaluated by a pathologist without the knowledge of the identity of the samples using a light microscope. Histopathological scores were assigned based on criteria as previously described. (Jacobson et al., Am. J. Physiol., 294 (2008) G295.) Scoring was performed based on severity of epithelial injury (graded 0-3, from absent to mild including superficial epithelial injury, moderate including focal erosions, and severe including multifocal erosions), the extent of inflammatory cell infiltrate (graded 0-3, from absent to transmural), and goblet cell depletion (0-1). For each tissue, a numerical score was assigned in a blinded manner to prevent bias. Scores from each tissue section group were averaged to obtain a mean histopathological score. Crypt heights were measured using ImageJ software, with 10 measurements taken in distal colons of each mouse. Only well-oriented and intact crypts were measured. Tissue sections were stained for cleaved caspase 3 (CC3) and macrophage infiltration (CD68). CC3 positive cells in the entire colon roll were counted at 20× magnification. Results were expressed as mean CC3 positive cells per entire colon roll. CD68 positive cells were counted in five randomly chosen areas in the colon roll at high power field (HPF). Results were expressed as mean of CD68 positive cells per HPF.

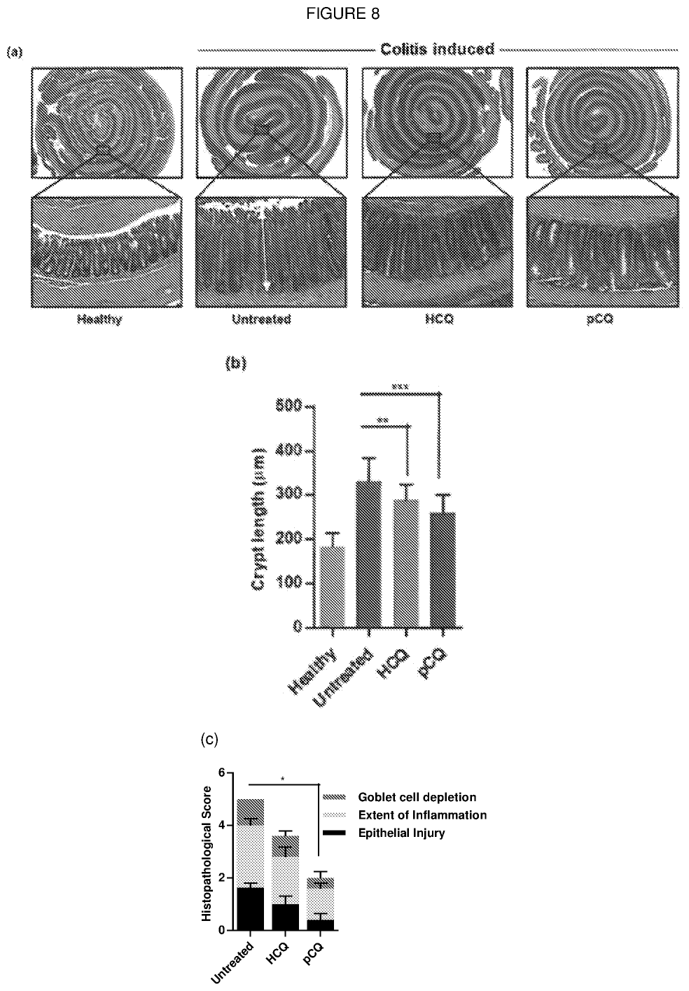

The local colon accumulation, limited systemic exposure, and low liver distribution of pCQ provided strong rationale for the testing of its anti-inflammatory activity in colitis. A therapeutic efficacy study was designed to assess whether restricting the distribution of pCQ to the GI tract preserves the activity of HCQ. The mice with colitis were treated (every other day) with oral gavage of pCQ and HCQ. Histological changes in the colon were examined following animal sacrifice on day 14. As shown in FIG. 8, untreated animals showed superficial epithelial damage, marked reduction in the goblet cell population, mucin depletion, inflammatory cell infiltration and crypt hyperplasia. Treatment with both, pCQ and HCQ, reduced the colon inflammation and eased the epithelial injury (FIG. 8a). Statistically significant reduction of the histological damage score was observed in animals treated with pCQ (FIG. 8c). Both treatments significantly reduced the colon crypt length when compared with the untreated group (FIG. 8b). These findings support previous reports showing the efficacy of chloroquine in DSS-induced model of colitis and human patients.

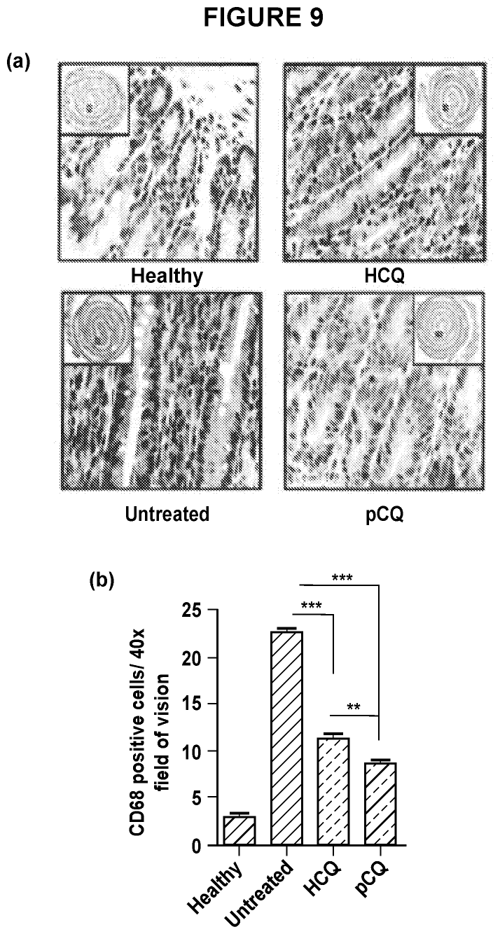

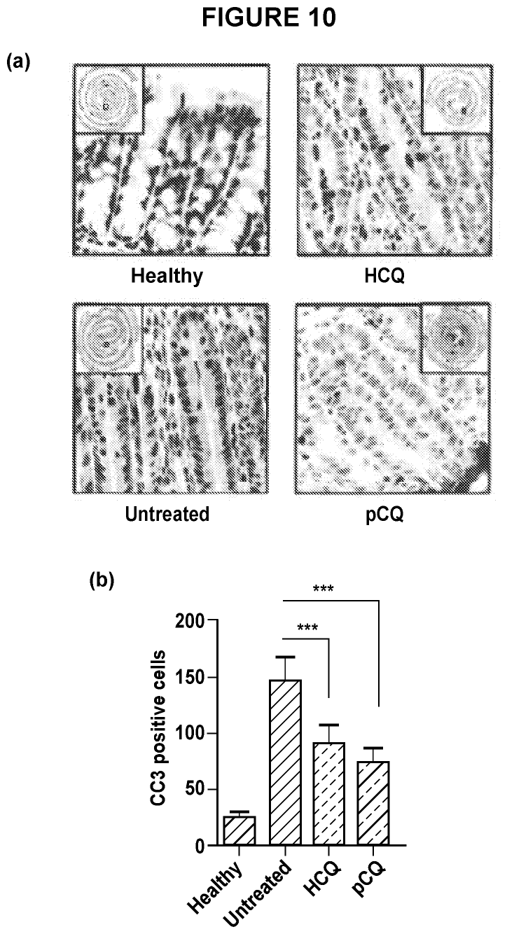

To assess the immunohistochemical changes that occur during inflammation, a study was conducted of the effect of pCQ treatment on the expression of two markers (CD68, CC3) that are commonly measured in reported in vivo IBD studies. CD68 is a transmembrane glycoprotein specifically expressed by monocytes and macrophages. CD68 plays an important role in macrophage homing to tissues and is used to study macrophage infiltration in the inflamed colon. Colon sections from patients with active IBD have significantly higher macrophage infiltration than healthy subjects. Elevated infiltration of CD68+macrophages was observed in the colons of untreated mice with colitis (FIG. 9a). Treatment with pCQ and HCQ showed statistically significant reduction in the macrophage infiltration, with pCQ outperforming HCQ (FIG. 9b). CD68+macrophages have different roles in UC and CD, but they massively infiltrate throughout the inflamed colon and targeting CD68 to reduce macrophage infiltration is a potential therapeutic strategy in IBD. Based on prior work which showed a broad ability of pCQ to inhibit migration and invasion of cells, and without wishing to be bound by theory, it is proposed that reduction of macrophage infiltration could be a possible mechanism by which pCQ is exerting its anti-inflammatory activity. Apoptosis was next evaluated as a marker for epithelial cell injury. CC3 immunostaining was used assess the apoptotic cells in the colon epithelium and it was found that pCQ treatment showed statistically significant reduction in the number of apoptotic epithelial cells when compared with the untreated group (FIG. 10). This observation combined with the decrease in the crypt length demonstrates the amelioration of epithelial cell injury by pCQ.

### Example 7—Real-Time PCR (RT-PCR)

Colon samples from the therapeutic study were stored in RNAlater™ (Thermo Fisher Scientific Inc.) at 4° C. for 48 hours to allow sufficient time for tissue penetration followed by removal of excess solution. The tissues were then stored at −80° C. until further processing. Stored frozen tissues were homogenized in TRIzol™ (Thermo Fisher Scientific Inc.) reagent using TissueLyser II (Qiagen) and mRNA was isolated from the homogenized tissues according to manufacturer's protocol. The extracted mRNA was quantified using Nanodrop Onec UV-Vis spectrophotometer (Thermo Fisher Scientific Inc.). The cDNA was synthesized from the mRNA using High-Capacity cDNA Reverse Transcription Kit with RNase Inhibitor per the manufacturer's protocol (Thermo Fisher Scientific Inc.). A volume corresponding to 1 μg of RNA as determined by UV spectrometer was used for cDNA synthesis. Synthesized cDNA was stored at −20° C. until further use. RT-PCR was carried out using the synthesized cDNA from colon tissue samples to determine the levels of mRNA of the target genes. Healthy and untreated colons were used as controls. cDNA was mixed with 0.2 μM of primer pair of gene of interest (Table 1) and iTaq™ Universal SYBR® Green Supermix (Biorad) into an optical reaction tube (Qiagen). The RT-PCR reaction was carried out in Rotor-Gene Q 2plex thermal cycler (Qiagen) using the following cycle program: 95° C. for 3 minutes; 40 cycles 60° C. for 30 seconds. Results obtained from the RT-PCR were analyzed by Ct method to determine the fold change in gene expression. Primer sequences for the RT-PCR are given in Table 6, below.

TABLE 6

| mRNA targets | Primer sequence (5′-3′) | |
|---|---|---|
| TNF-α | F | CATGAGCACAGAAAGCATGATC |
| | | (SEQ ID NO: 1) |
| | R | CCTTCTCCAGCTGGAAGACT |
| | | (SEQ ID NO: 2) |
| IL-6 | F | ATGGATGCTACCAACTGGAT |
| | | (SEQ ID NO: 3) |
| | R | TGAAGGACTCTGGCTTTGTCT |
| | | (SEQ ID NO: 4) |
| IL-1β | F | CAACCAACAAGTGATATTCTCCATG |
| | | (SEQ ID NO: 5) |
| | R | GATCCACACTCTCCAGCTGCA |
| | | (SEQ ID NO: 6) |
| IL-2 | F | TGAGCAGGATGGAGAATTACAGG |
| | | (SEQ ID NO: 7) |
| | R | GTCCAAGTTCATCTTCTAGGCAC |
| | | (SEQ ID NO: 8) |

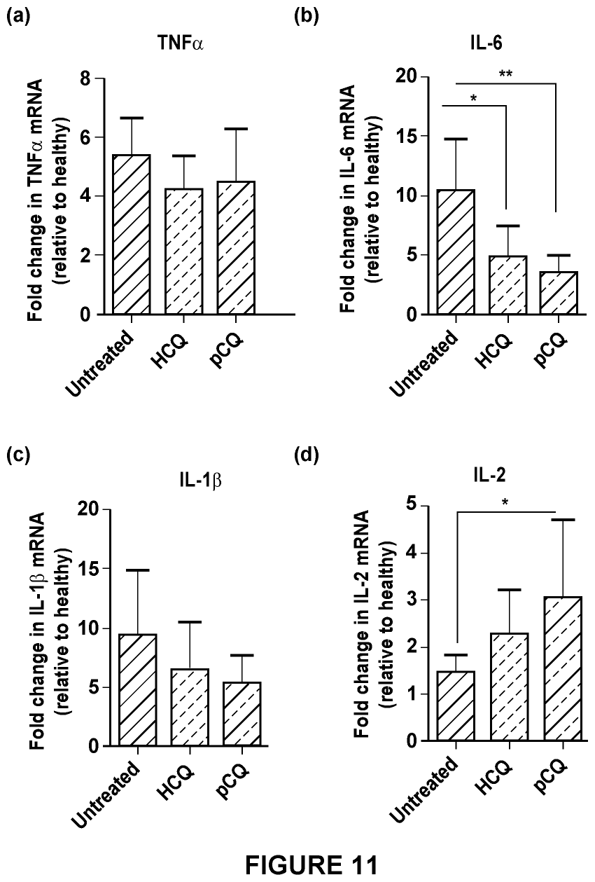

Following histological evaluation, the mechanisms of anti-inflammatory activity of pCQ were explored. Upregulation of pro-inflammatory cytokines is a hallmark of IBD and lowering local levels of cytokines has been shown to reduce colon inflammation. To investigate how pCQ treatment changes the cytokine expression profile, mRNA levels of selected pro-inflammatory cytokines were measured in the colons after oral administration of seven doses of pCQ over 14 days. Although inhibition of TNFα is a well-established approach in the treatment of IBD and TNFα expression was 5-times higher in the untreated group, we observed no significant reduction in the colon TNFα expression after treatment with pCQ (FIG. 11a). Expression levels of IL-6, IL-113 and IL-2 were also measured in the colon. Initial studies indicated that IL-6 was highly upregulated in the C. rodentium model. Here, statistically significant reduction of IL-6 expression was observed by both pCQ and HCQ (FIG. 11b). Similar effects of pCQ and HCQ treatments are also seen in the expression of IL-1l3 (FIG. 11c).

In contrast to IL-6 and IL1β, both treatments resulted in upregulated IL-2 expression (FIG. 11d). Statistically significant difference was observed between IL-2 levels in untreated control and pCQ groups. IL-2 knockout mice are an often-used animal model of IBD and there has been a reported clinical trial which investigated subcutaneously administered IL-2 as a way of enhancing regulatory T cells in IBD patients to reduce inflammation [50]. Based on these findings, upregulation of IL-2 may represent an interesting direction in the mechanistic studies of pCQ anti-inflammatory activity.

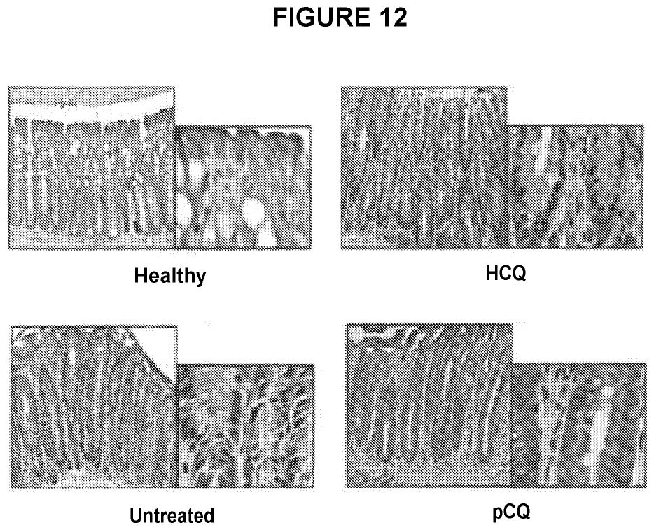

To further elucidate the mechanism of pCQ action, the expression of STAT3, which is a downstream target of IL-6 with a known importance in the development of IBD, was observed. As shown in FIG. 12, STAT3 was upregulated in the untreated mice and pCQ inhibited STAT3 more efficiently than HCQ. IL-6 and STAT3 have been targets of many mechanistic as well as clinical and preclinical studies. The IL-6 and STAT3 pathways promote immune response by increasing the CD4+ T-cell migration into the inflamed colon, which consequently increases the migration of other immune cells to the inflamed areas. Accumulated evidence suggests that activation of IL-6 and STAT3 is an important inflammatory event in the development of IBD. Hence, inhibiting IL-6 and STAT3 signaling pathways represents a possible mechanism for the observed anti-inflammatory activity of pCQ. Overall, these observations pointed out that the therapeutic activity of pCQ seemed to be an effect of restoring the colonic immune imbalance that occurs in IBD.

In view of the many possible embodiments to which the principles of the disclosure may be applied, it should be recognized that the illustrated embodiments are only examples and should not be taken as limiting the scope of the invention.

What is claimed:

1. A copolymer comprising a plurality of a first monomer and a plurality of a second monomer,
wherein chloroquine, hydroxychloroquine, or a chloroquine analog is appended to at least a portion of the plurality of the first monomer.

2. The copolymer of claim 1, wherein chloroquine, hydroxychloroquine, or a chloroquine analog is appended to all of the first monomer.

3. The copolymer of claim 1 or 2, comprising chloroquine.

4. The copolymer of claim 1 or 2, comprising hydroxychloroquine.

5. The copolymer of claim 1 or 2, comprising a chloroquine analog.

6. The copolymer of claim 5, wherein the chloroquine analog is hydroxychloroquine, methacryloyl chloroquine, quinacrine, 8-hydroxyquinoline, primaquine, sontoquine, azidoquine, or methacryloyl triazole chloroquine.

7. The copolymer of claim 6, wherein the chloroquine analog is methacryloyl chloroquine.

8. The copolymer of any one of claims 1 to 7, wherein at least one of the first monomer and the second comprises an acrylate, an acrylamide, or an alkene.

9. The copolymer of any one of claims 1 to 8, wherein the first monomer having chloroquine appended thereto comprises 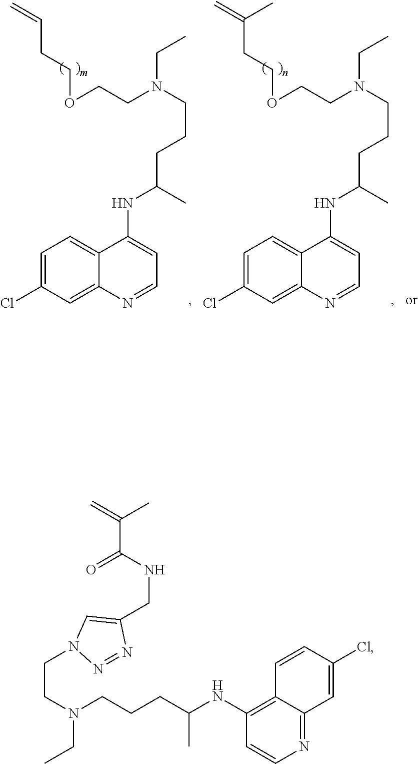
wherein m and n each are independently an integer from 0 to 10.

10. The copolymer of claim 9, wherein the first monomer having chloroquine appended thereto comprises

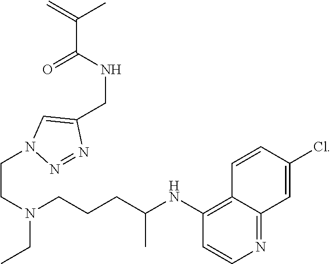 [SMILES: C=C(C)C(=O)NCc1cn(CCN(CC)CCCC(C)Nc2ccnc3cc(Cl)ccc23)nn1]

11. The copolymer of any one of claims 1 to 10, wherein the second monomer comprises N-(2-hydroxypropyl)methacrylamide (HPMA).

12. The copolymer of any one of claims 1 to 11, further comprising a polymer block.

13. The copolymer of claim 12, wherein the polymer block comprises poly(lactide-co-glycolide) (PLGA), polylactic acid (PLA), polycaprolactone (PCL), other polyesters, poly(propylene oxide), poly(1,2-butylene oxide), poly(n-butylene oxide), poly(tetrahydrofurane), and polystyrene; glyceryl esters, polyoxyethylene fatty alcohol ethers, polyoxyethylene sorbitan fatty acid esters, polyoxyethylene fatty acid esters, sorbitan esters, glycerol monostearate, polyethylene glycols, polypropyleneglycols, cetyl alcohol, cetostearyl alcohol, stearyl alcohol, aryl alkyl polyether alcohols, polyoxyethylene-polyoxypropylene copolymers, poloxamines, cellulose, methylcellulose, hydroxylmethylcellulose, hydroxypropylcellulose, hydroxypropylmethylcellulose, polysaccharides, starch and their derivatives, hydroxyethylstarch, polyvinyl alcohol (PVA), polyvinylpyrrolidone phospholipids, amphiphilic lipids, 1,2-dialkylglycero-3-alkylphophocholines, 1,2-distearoyl-sn-glecro-3-phosphocholine (DSPC), 1,2-distearoyl-sn-glycero-3-phosphoethanolamine-N-[carboxy(polyethylene glycol) (DSPE-PEG), dimethylaminoethanecarbamoyl cheolesterol (DC-Chol), N-[1-(2,3-Dioleoyloxy)propyl]-N,N,N-trimethylammonium (DOTAP), alkyl pyridinium halides, quaternary ammonium compounds, lauryldimethylbenzylammonium, acyl carnitine hydrochlorides, dimethyldioctadecylammonium (DDAB), n-octylamines, oleylamines, benzalkonium, cetyltrimethylammonium, chitosan, chitosan salts, poly(ethylenimine) (PEI), poly(N-isopropyl acrylamide (PNIPAM), and poly(allylamine) (PAH), poly (dimethyldiallylammonium chloride) (PDDA), alkyl sulfonates, alkyl phosphates, alkyl phosphonates, potassium laurate, triethanolamine stearate, sodium lauryl sulfate, sodium dodecylsulfate, alkyl polyoxyethylene sulfates, alginic acid, alginic acid salts, hyaluronic acid, hyaluronic acid salts, gelatins, dioctyl sodium sulfosuccinate, sodium carboxymethylcellulose, cellulose sulfate, dextran sulfate and carboxymethylcellulose, chondroitin sulfate, heparin, synthetic poly(acrylic acid) (PAA), poly (methacrylic acid) (PMA), poly(vinyl sulfate) (PVS), poly(styrene sulfonate) (PSS), esters and amides of poly(methacrylic acid) and poly(acrylic acid) (e.g., alkylmethacrylates—methyl, ethyl, propyl, butyl) bile acids and their salts, cholic acid, deoxycholic acid, glycocholic acid, taurocholic acid, glycodeoxycholic acid, a derivative thereof, or a combination thereof.

14. The copolymer of claim 12 or 13, wherein the polymer block comprises styrene.

15. The copolymer of claim 14, wherein the second monomer comprises HPMA and the polymer block comprises polystyrene.

16. The copolymer of any one of claims 1 to 15, wherein the chloroquine, hydroxychloroquine, or chloroquine analog is attached to the first monomer via a bond cleavable from the polymer block under physiological conditions.

17. The copolymer of claim 16, wherein the chloroquine, hydroxychloroquine, or chloroquine analog is attached to the first monomer via an amide bond, an ester bond, a 5-10 membered heterocycle, or a peptide bond.

18. The copolymer of any one of claims 1 to 17, wherein the chloroquine, hydroxychloroquine, or chloroquine analog is attached to the first monomer via a bond not cleavable from the polymer block under physiological conditions.

19. The copolymer of claim 18, wherein the chloroquine, hydroxychloroquine, or chloroquine analog is attached to the first monomer via a triazolyl ring.

20. The copolymer of any one of claims 1 to 17, wherein the chloroquine, hydroxychloroquine, or chloroquine analog is attached to the first monomer via a bond cleavable from the polymer block under physiological conditions.

21. The copolymer of claim 1 having a structure of: 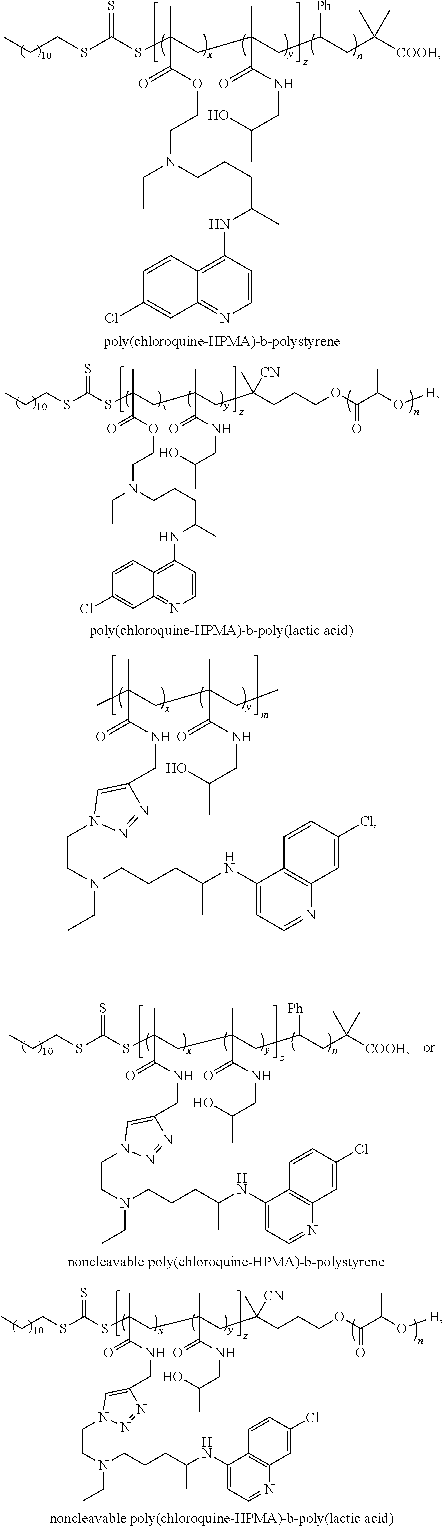
each x is independently an integer of 1 to 200;
each y is independently an integer of 0 to 500;
each z is independently an integer of 1 to 500;
each m is independently an integer of 1 to 500 and
each n is independently an integer of 1 to 10,000.

22. The copolymer of any one of claims 1 to 21, further comprising a crosslinker.

23. The copolymer of claim 22, wherein the crosslinker is cleavable under physiological conditions.

24. The copolymer of claim 23, wherein the crosslinker is a reactive oxygen species—(ROS)—sensitive linker.

25. The copolymer of claim 24, wherein the crosslinker is an aromatic azo linker degradable by colonic azo-reductases.

26. The copolymer of claim 25, wherein the ROS-sensitive linker comprises a thioketal crosslinker, organochalcogen, arylboronic esters, thioether, vinyldithioether, aryl oxalate ester, or ferrocene.

27. The copolymer of any one of claims 21 to 24, wherein the crosslinker comprises

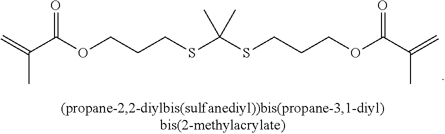 [SMILES: C=C(C)C(=O)OCCCSC(C)(C)SCCCOC(=O)C(=C)C]

28. A bead, microparticle or nanoparticle comprising a plurality of the copolymer of any one of claims 1 to 27.

29. The bead, microparticle or nanoparticle of claim 28, comprising a polymer of any one of claims 12 to 26.

30. The bead, microparticle or nanoparticle of claim 27 or 28, further comprising a therapeutic agent.

31. The bead, microparticle or nanoparticle of claim 30, wherein the therapeutic agent comprises an anti-inflammatory compound, an immune system suppressor, an antibiotic, or a combination thereof.

32. The bead, microparticle or nanoparticle of claim 31, wherein the anti-inflammatory compound comprises an aminosalicylate, a corticosteroid, cyclosporine, small interfering RNA (siRNA), or an antibody.

33. The bead, microparticle or nanoparticle of claim 32, wherein the antibody or siRNA is an anti-TNFα antibody or anti-TNFα siRNA.

34. The bead, microparticle or nanoparticle of any one of claims 28 to 33, further comprising a targeting ligand.

35. The bead, microparticle or nanoparticle of claim 34, wherein the targeting ligand comprises a lectin or mannose.

36. A method of treating inflammatory bowel disease (IBD) comprising administering to a patient in need thereof a therapeutically effective amount of the copolymer of any one of claims 1 to 27, or the bead, microparticle or nanoparticle of any one of claims 28 to 35.

37. The method of claim 36, wherein IBD comprises ulcerative colitis, Crohn's disease, irritable bowel syndrome, amebic colitis, acute self-limiting colitis, or colitis.

38. The method of claim 37, wherein IBD comprises colitis.

39. A method of treating colon cancer comprising administering to a patient in need thereof a therapeutically effective amount of the copolymer of any one of claims 1 to 27, or the bead, microparticle or nanoparticle of any one of claims 28 to 35.

40. The method of claim 39, wherein the administering comprises oral administration.

41. A method of making the copolymer of any one of claims 1 to 27, comprising:
admixing a plurality of the first monomer and the second monomer under conditions to polymerize the first monomer and the second monomer to form the copolymer, optionally in the presence of a
polymerization initiator, a chain transfer agent, or both.

42. A method of making the copolymer of any one of claims 1 to 27, comprising:
admixing a plurality of a first monomer precursor and the second monomer under conditions to polymerize the first monomer precursor and the second monomer to form a copolymer precursor, optionally in the presence of a polymerization initiator, a chain transfer agent, or both; and
reacting the copolymer precursor with chloroquine or a chloroquine analog to append the chloroquine or the chloroquine analog to the copolymer precursor to form the copolymer.

43. The method of claim 42, wherein the first monomer precursor comprises an alkyne moiety, and the chloroquine or chloroquine analog comprises an azide moiety, and the reacting comprises formation of a triazolyl between the alkyne and the azide to append the chloroquine or the chloroquine analog.

44. The method of any one of claims 41 to 43, comprising admixing in the present of the polymerization initiator.

45. The method of claim 44, wherein the polymerization initiator comprises azobisisobutyronitrile (AIBN).

46. The method of any one of claims 41 to 45, comprising admixing in the presence of the chain transfer agent.

47. The method of claim 46, wherein the chain transfer agent comprises 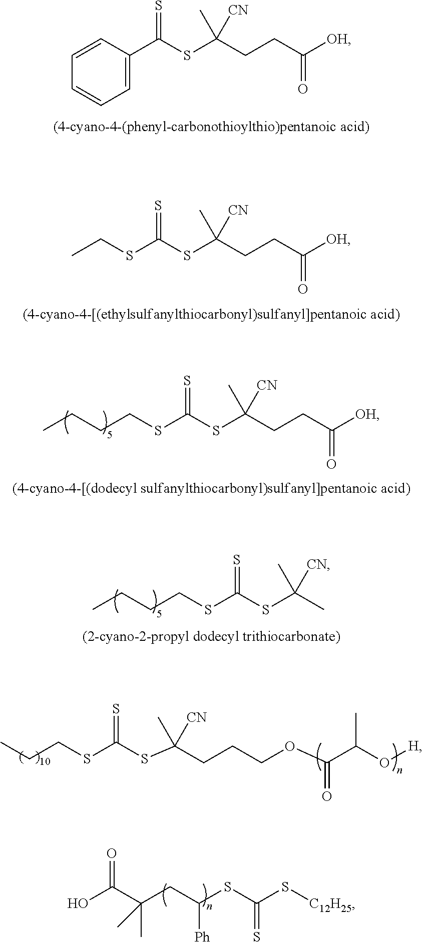
or a mixture thereof.

48. The method of claim 47, wherein the chain transfer agent comprises 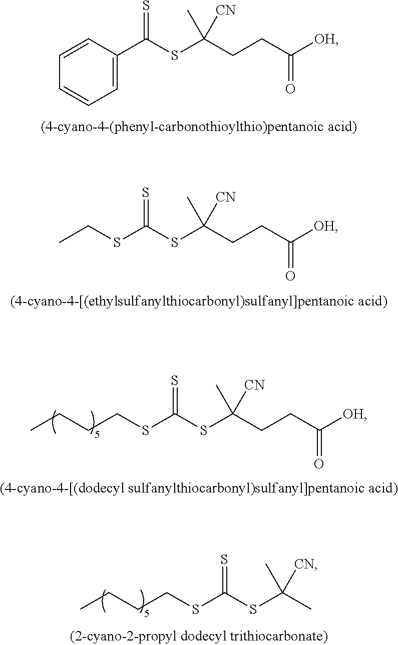
or a mixture thereof.
